(19) United States
(12) Patent Application Publication
(10) Pub. No.: US 2006/0172357 A1
(43) Pub. Date: Aug. 3, 2006

(54) Detecting human antibodies in non-human serum

(75) Inventors: Jihong Yang, Foster City, CA (US); Valerie Elizabeth Quarmby, Portola Valley, CA (US)

Correspondence Address: MERCHANT & GOULD PC, P.O. BOX 2903, MINNEAPOLIS, MN, 55402-0903, US

(73) Assignee: Genentech, South San Francisco, CA (US)

(21) Appl. No.: 11/323,325

(22) Filed: Dec. 30, 2005

Related U.S. Application Data
(60) Provisional application No. 60/640,968, filed on Dec. 31, 2004.

(51) Int. Cl. G01N 33/537; G01N 33/543

(52) U.S. Cl. 435/7.92

(57) ABSTRACT

The present invention provides for the quantification of a human, human-chimeric, humanized antibody, or a fragment thereof, without the necessity of using a target-specific molecule. More particularly, the invention relates to a quantification assay that includes the step of blocking non-specific binding sites of a capture reagent with a blocking buffer containing a non-human mammalian globulin, such as bovine gamma globulin (BGG).

### BACKGROUND OF THE INVENTION

Antibodies, including humanized monoclonal antibodies, have been widely studied for their potential therapeutic application in humans. During early development of a therapeutic antibody, efficacy and safety of the molecule is studied. Commonly, such studies involve administration of the antibody to a non-human species, such as a non-human primate. Body fluid obtained from the non-human species, for example, serum, is analyzed for the presence and concentration of the antibody of interest. To carry out this analysis, a sensitive serum pharmacokinetic (PK) assay capable of specifically detecting and quantifying the antibody in the body fluid of the non-human species is required.

In general, pharmacokinetic assays useful to determine the concentration of a target molecule in a biological matrix such as serum requires the use of one or more target-specific molecules, particularly when the target molecule is a humanized IgG present in the serum of a non-human primate such as a cynomolgus monkey. Biological matrices tend to cause a high assay background, due to non-specific interactions of the matrix components in the assay. Analysis of a first species antibody present in biological fluid of a closely-related second species can be particularly difficult because of high sequence identity between the IgGs of the two species. For example, sequence identity between cynomolgus monkey IgG and human IgG is reported to be about 83% for Kappa constant domains (C_{κ}), 88-99% for Kappa variable domains (V₇₈) framework regions, 93% for the heavy chain variable domain (V_{H}) framework regions, and 93-95% for the heavy chain constant domain (C_{H}). See, for example, Lewis et al., 1993, Developmental and Comparative Immunology, 17: 549-60; D'Ovidio et al., 1994, Folia Primatol 63:221-25; Pace et al., 1996, Immunol. Lett. 50:139-42. Circulating levels of cynomolgus IgG are commonly in the range of 10 to 16 mg/ml, much higher than the levels of a target human antibody present in cynomolgus serum during analysis, that may be as low as 20 ng/ml (Biagini et al, 1988, Laboratory Animal Science, 38:194; Tryphonas et al, 1991, J Med Primatol, 20:58).

An exemplary human antibody is rhuMAb2H7, a fully humanized monoclonal antibody derived from a murine precursor, 2H7, and belonging to the human IgG1 family with a kappa light chain (Clark et al., 1985, Proc. Natl. Acad. Sci. USA. 82:1766-70). The rhuMAb2H7 antibody is directed against the extracellular domain of the CD20 antigen, expressed on both normal and malignant B cells (Stashenko et al., 1980, J. Immunol. 125:1678-85; Clark et al., 1989, Adv. Cancer Res. 52:81-149; Tedder et al., 1994, Immunol. Today 15:450-54; Tedder et al., 1988, J. Biol. Chem. 263:10009-10015; Riley et al., 2000, Semin. Oncol. 27:17-24).

B cell depleting reagents have been used successfully to treat malignant B cell-mediated cancers such as non-Hodgkin's lymphoma (McLaughlin et al., 1988, Oncology 12:1763-69); and chronic lymphocytic leukemia (Jensen et al., 1998, A. Ann Hematol 77:89-91; Gopal et al., 1999, J. Lab. Clin. Med. 134:445-50; von Schilling, 2003, Semin. Cancer Biol., 2003, 13:211-22. B-cells are also involved in autoimmune diseases such as rheumatoid arthritis (Dorner et al, 2003, Opin. Rheumatology 15, 246-52; Looney, 2002, Ann. Rheum. Dis. 61, 863-6; Shaw et al., 2003), systemic lupus erythematosus (Anolik et al., 2003, Current Rheum. Reports. 5, 350-6), and multiple sclerosis (Hafler, 2004, J Clin Invest. 113(6):788-94.

Binding of rhuMAb2H7 to the CD20 antigen results in depletion of B cells in vivo. (Vugmeyster et al., 2004, Int Immunopharmacol. 4(8):1117-24). Although the exact mechanism of B-cell depletion by rhuMAb2H7 is unknown, in vivo efficacy data, along with that from other anti-CD20 therapeutics, indicate that rhuMAb2H7 is a potential therapeutic for both B-cell mediated autoimmune disorders and for oncology indications.

During the early development of rhuMAb2H7, a first proof-of concept study was carried out in cynomolgus monkeys to assess the efficacy and safety of the molecule. A sensitive PK assay to detect and quantify rhuMAb2H7 in cynomolgus monkey serum was needed to support pharmacokinetic evaluations. The development of such an assay posed distinct challenges, including the lack of available target-specific molecules.

To address the challenges of specifically distinguishing humanized IgG from cynomolgus monkey IgGs, available assays for detecting humanized IgG molecules in cynomolgus monkey serum utilize one or more target-specific molecules. For example, in rhuMAb2H7 cynomolgus monkey pilot studies, an assay sensitivity of 20 ng/ml was needed. Such target-specific molecules were not readily available for binding to anti-CD20 antibodies.

To solve this problem, an alternative assay was developed. The present invention provides novel methods for quantifying molecules, for example antibodies, in biological matrices such as body fluids without the use of target-specific capture or detecting reagents. The disclosed assays have high sensitivity and are applicable to a wide range of molecules, including polypeptides such as antibodies. The disclosed assays are particularly useful in detecting a first animal species antibody when disposed in body fluid of a second animal species, even closely related species such as human IgG and non-human primate IgG.

### SUMMARY OF THE INVENTION

The present invention provides methods for determining the presence or amount of a first species molecule in the presence of similar molecules of a second species. Molecules, for example, antibodies of a first species, disposed in a biological matrix of a second species, such as serum or other body fluid, can be detected and quantitated using methods described herein. The sample to be assayed can be, for example, a first species antibody, such as a human, human chimeric, or humanized antibody, or an antigen-binding fragment thereof, disposed in body fluid of a second species, for example, a non-human species, including a closely related primate species such as cynomolgus monkeys. In one embodiment, the antibody fragment comprises a constant domain.

In general, it has now been discovered that addition of a mammalian globulin protein such as bovine gamma globulin (BGG) as a specific blocking agent in the blocking step of a ligand binding assay greatly improves assay sensitivity by reducing serum background and background variation in the assay. An additional step of preadsorbing capture reagent, for example, with the biological matrix of the second species (or a species closely related to the second species), also reduces assay background and further increases sensitivity of the assay.

In one embodiment, a sensitive assay to detect a human, humanized, or chimeric antibody, or fragment thereof, disposed in a non-human body fluid such as serum, generally comprises the following steps:
(1) applying a capture reagent to an assay surface;
(2) blocking non-specific binding sites of the capture reagent with a blocking buffer containing a non-human mammalian globulin such as bovine gamma globulin;
(3) reacting a sample with the blocked capture reagent; and
(4) detecting captured antibodies with a detection agent, for example, a detection agent capable of generating a detectable signal.

Each of the capture reagent and the detection agent can bind the molecule to be detected. The capture reagent and the detection agent can bind the same or a different ligand or epitope of the molecule to be detected. For example the capture reagent and the detection reagent can comprise, the same antibody.

The capture reagent can be preadsorbed with the biological matrix, for example, the body fluid of the second species, or that of a species closely related to the second species. In one embodiment, the first species is human, and the second species is a non-human species, for example, a non-human mammal, such as a primate, and can be, for example, monkey, bovine, porcine, equine, ovine, and the like. Closely related species are typically those within the same family, and can be within the same genus.

In one embodiment, the blocking buffer comprises a mammalian globulin as a non-specific blocking agent. In an assay to detect a human, humanized, or human chimeric antibody, for example, the blocking buffer comprises a non-human mammalian globulin such as bovine gamma globulin (BGG), mouse IgG, rabbit IgG, or donkey IgG. Where a bridging ELISA format is used, mammalian globulin can be present in the blocking buffer, but not in the sample buffer and/or detection buffer. Where a direct ELISA format is used, mammalian globulin can be present in each of the blocking buffer, sample buffer, and detection agent buffer.

The assay methods described herein can be used to detect and/or quantitate a molecule of a first species, including polypeptides such as antibodies, for example human, human chimeric, and humanized antibodies, and fragments thereof, such as a Fab fragments, and the like, when the molecules, for example, antibodies or antibody fragments, are disposed in a biological matrix of a second species, for example, in body fluid such as non-human serum. Recombinant, humanized monoclonal antibodies such as the anti-HER2 antibody HERCEPTIN®, the anti-CD20 antibody rhuMAb2H7, and the like, can be detected and/or quantitated in non-human primate serum with high sensitivity and without use of target-specific capture reagents, by use of the assay methods disclosed herein.

### BRIEF DESCRIPTION OF THE FIGURES

FIG. 1 is a graph showing a rhuMAb2H7 standard curve of a target-specific assay for detecting rhuMAb2H7, using full-length CD20 antigen molecule as a target-specific capture reagent.

FIG. 2 is a bar graph showing signal to noise ratios in ELISA assays for detecting rhuMAb2H7 in cynomolgus monkey serum. The assays were designed to test various buffer ingredients and buffers in diluted and undiluted form.

FIG. 3 is a graph showing standard curves of rhuMAb2H7 (anti-CD20), Avastin™ (anti-VEGF), and Raptiva® (anti-CD11a) antibodies generated in an assay system using BGG in the blocking buffer, and not in the sample diluent or detection buffers.

FIG. 4 is a graph showing standard curves for rhuMAb2H7, Xolair® (anti-IgE), and Herceptin® (anti-HER2) antibodies generated in an assay system using BGG in the blocking buffer, and not in the sample diluent or detection buffers.

FIG. 5 is a graph showing standard curves for Rituxan® antibody (anti-CD20) generated in an assay system using BGG in the blocking buffer, and not in the sample diluent or detection buffers.

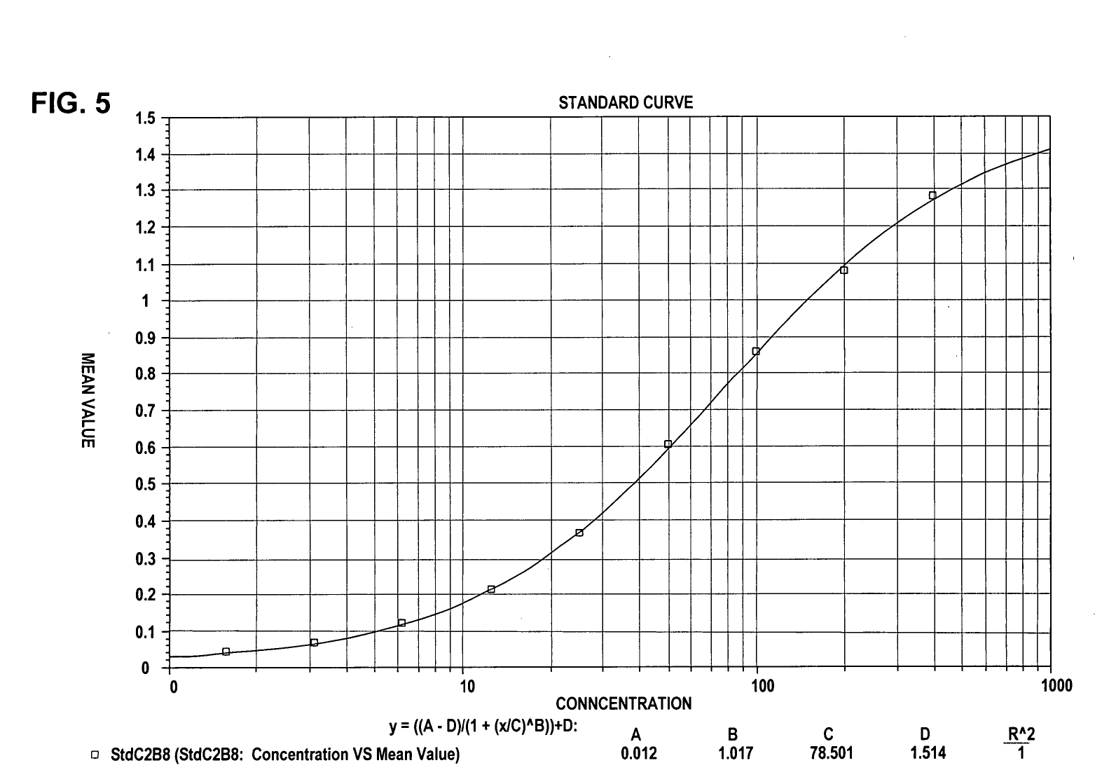

### DETAILED DESCRIPTION OF THE PREFERRED EMBODIMENTS

I. Definitions

“Assay surface” is meant to encompass any surface useful to immobilize a capture reagent for use in the assay systems described herein. The assay surface may comprise an inert support or carrier that is essentially water insoluble and useful, for example, in immunoassays, and including supports in the form of surfaces, particles, porous matrices, and the like. Specific assay surfaces include, for example, microtitre plates, chromatography resin, sensor chips, and the like.

The term “capture reagent” refers to a reagent capable of binding and capturing a target molecule or analyte to be detected in a sample. Typically, the capture reagent is immobilized, for example on an assay surface, for example, a solid substrate, such as a microparticle or bead, microtiter plate, column resin, and the like. The capture reagent is a molecule that binds the molecule to be detected and quantitated in the assay (the target molecule or analyte). When the molecule to be detected is an antibody, the capture reagent can be, for example, an antigen, soluble receptor, antibody, antibody fragment, a mixture of different antibodies, and the like, that binds the target antibody.

The term “detecting” is used in the broadest sense to include both qualitative and quantitative measurements of a specific molecule, herein measurements of a specific target molecule such as a humanized antibody. The assay methods described herein can be used to identify the mere presence of a target molecule in a sample. The assay methods can also be used to quantify an amount of a target molecule in a sample. The assay methods can also be used to determine the relative binding affinity of a target molecule for its binding partner, for example, the relative binding affinity of an antibody for its ligand.

The term “detecting agent” refers to an agent that detects the target molecule of an assay, either directly via a label, such as a fluorescent, enzymatic, radioactive, or chemiluminescent label, and the like, that can be linked to the detecting agent, or indirectly via a labeled binding partner, such as an antibody or receptor that specifically binds the detecting agent. Direct and indirect detecting agents are known. Examples of detecting agents include, but are not limited to, an antibody, antibody fragment, soluble receptor, receptor fragment, and the like.

In one embodiment, the detecting agent can be expressed on the coat of a phage. In one embodiment, the detecting agent is a direct label, for example, an antibody conjugated to horseradish peroxidase (HRP). In one embodiment, the detecting agent is indirect and comprises a biotin-conjugated antibody and a streptavidin-HRP conjugate.

The term “label,” as used herein, includes agents that amplify a signal produced by a detecting agent. The label may be a radiologic, photoluminescent, chemiluminescent (such as HRP), or electrochemiluminescent chemical moiety, an enzyme that converts a colorless substrate into a colored product, and the like. Examples of enzyme labels include, but are not limited to, horseradish peroxidase, alkaline phosphatase, β-D-galactosidase, glucoamylase, glucose oxidase, acetylcholine esterase, glutamate decarboxylase, catalase, urease, adenosine electrode, lysozyme, malate dehydrogenase, glucose-6-phosphate dehydrogenase, hexokinase, β-amylase, and phospholipase C. Examples of fluorescent labels include, but are not limited to, coumarin derivatives, fluorescein, rhodamine, europium, phycoerythrin, samarium, terbium, and umbelliferone. Examples of luminescent labels include, but are not limited to, acridinium ester and isoluminol derivatives. Other labels may also be used, including, for example, ligand labels such as avidin or biotin derivatives, particle labels, radioisotopic labels, vesicle labels, colloidal metal labels, and spin labels such as nitroxide radical.

The term “antibody” herein is used in the broadest sense and specifically includes intact monoclonal antibodies, polyclonal antibodies, chimeric antibodies, humanized antibodies, multispecific antibodies (e.g., bispecific antibodies) formed from at least two antibodies, and antibody fragments exhibiting the desired biological activity. The antibody may be natural or synthetic, and may include amino acid variations such as substitutions that do not ablate the antibody's binding activity. A “chimeric antibody” contains at least a portion of its heavy and/or light chain that differs from the remainder of the antibody in species or in antibody class or subclass and includes fragments that exhibit the desired biological activity (U.S. Pat. No. 4,816,567; Morrison et al., 1984, Proc. Natl. Acad. Sci USA, 81:6851-6855).

“Humanized” forms of non-human (for example, murine) antibodies are chimeric antibodies that contain minimal sequence derived from non-human immunoglobulin. In general, humanized antibodies are human immunoglobulins (recipient antibody) wherein residues from the antibody's hypervariable region (Complementarity Determining Regions (CDRs) defined by sequence according to Kabat et al., 1991, Sequences of Proteins of Immunological Interest, 5th Ed. Public Health Service, National Institutes of Health, Bethesda, Md. or hyperviariable loops (HVLs) defined structurally according to Chothia and Lesk, 1987, J. Mol. Biol. 196:901-917) of the recipient are replaced by residues from a corresponding hypervariable region of a non-human species (donor antibody) such as mouse, rat, rabbit, or nonhuman primate having the desired specificity, affinity, and capacity. In some instances, specific variable domain framework region (FR) residues of the human immunoglobulin are replaced by corresponding non-human residues. Furthermore, humanized antibodies may contain residues that are not found in the recipient antibody or in the donor antibody. Such modifications are generally made to further refine antibody performance. In general, the humanized antibody will comprise substantially all of at least one heavy or light chain variable domain, and typically comprise a heavy and a light chain variable domain, in which all or substantially all of the hypervariable loops or CDRs correspond to those of a non-human immunoglobulin and all or substantially all of the FR regions are those of a human immunoglobulin sequence or a human consensus sequence. The humanized antibody optionally contains at least a portion of an immunoglobulin constant region (Fc), typically that of a human immunoglobulin. See, for example, Jones et al., 1986, Nature 321:522-525; Riechmann et al., 1988, Nature 332:323-329; and Presta, 1992, Curr. Op. Struct. Biol. 2:593-596.

A “fragment” of an antibody, including fragments of chimeric and humanized antibodies, contains a portion of an intact antibody, for example, an antigen-binding or variable region of the intact antibody. Examples of antibody fragments include Fab, Fab′, F(ab′)₂, and Fv fragments, diabodies, linear antibodies, single-chain antibody molecules, multispecific antibodies formed from antibody fragment(s), and the like.

“Closely related species” are those within a same family, and preferably within a same genus.

“Primate” is construed to mean any of an order of mammals comprising humans, apes, baboons, chimpanzees, monkeys, and related forms, such as lemurs and tarsiers. Monkeys include, for example, rhesus, cynomolgus, and African green.

II. Modes for Carrying Out the Invention

The invention provides accurate and highly sensitive screening methods for detecting and/or quantifying a target molecule of interest, such as a human, human-chimeric, or humanized antibody, or a fragment of such antibodies. The assay methods of the invention provide sensitive screening of target molecules, including human antibodies, present in a complex biochemical medium such as non-human serum, without the need of a target-specific molecule as a capture reagent.

Generally, one assay method according to the invention comprises the following steps:
(1) applying a first species capture reagent to an assay surface;
(2) blocking non-specific binding sites of the capture reagent with a blocking buffer containing a globulin of a second species;
(3) reacting the blocked capture reagent with a sample to capture any of the target molecule, for example, human, humanized, or chimeric antibody, present in the sample, for example, in a non-human serum such as monkey serum, and
(4) detecting the captured target molecule antibody with a detection agent.

The invention is based in part upon the discovery that the use of a non-human mammalian globulin in the assay blocking buffer can result in a quantitative assay for human, humanized, or chimeric antibodies that exhibits a relatively low background, as well as relatively low background variation between samples, while maintaining a high sensitivity for the target molecule. It is expected that the methods of the invention can be useful for analysis of human or non-human (first species) target molecules in the presence of a second species biological matrix.

A. Capture Reagent

A choice of capture reagent to be used in the methods described herein is generally determined by the target molecule to be quantified or detected. The capture reagent is chosen for its ability to bind and capture a target molecule from a sample. When the target molecule is an antibody, for example, the capture reagent can be an antibody, such as an anti-human IgG. In one embodiment, for example, quantitating a humanized antibody in non-human serum, the capture reagent can be sheep or goat anti-human IgG, for example. The capture reagent can be applied to any suitable assay surface, such as a microtiter plate, chromatography resin, sensor chip, and the like.

B. Preadsorption of the Capture Reagent

Biological matrices, such as non-human body fluids, tend to produce a high and undesirable assay background due to nonspecific protein interactions.

In one embodiment, a capture reagent is preadsorbed with a potentially interfering material from the biological matrix. For example, in a method for analyzing a human antibody present in monkey serum, the capture reagent is preabsorbed with a non-human body fluid such as non-human serum, to reduce nonspecific interactions when the sample body fluid is contacted with the capture reagent. Preadsorption of the capture reagent can serve to reduce assay background compared to capture reagent that has not been preadsorbed.

The capture reagent can be preadsorbed with body fluid, such as serum, from a second species, e.g., non-human species or a species closely related to the second species. For example, for analysis of a humanized antibody (first species) disposed in monkey serum, the body fluid is monkey serum, and the capture reagent is a binding ligand preadsorbed with monkey serum (second species) or serum of a closely related species, such as a different primate, to reduce nonspecific interactions.

C. Blocking Buffers

Capture reagents suitable for use in the methods described herein have the potential to produce non-specific interactions with components of the sample other than the desired target molecule (e.g., antibody) to be detected and/or quantitated. Before reacting a capture reagent with a sample containing a target molecule (e.g. antibody) to be quantitated, non-specific binding sites of the capture reagent may be blocked through use of a blocking buffer. A blocking buffer serves to bind and saturate non specific binding sites and prevent unwanted binding of free ligand to excess binding sites on the assay surface.

Various blocking buffers are known, and can include as active blocking components, for example, bovine serum albumin (BSA), gelatin, Superblock (Pierce, Rockford, Ill.), Casein (Pierce), and the like. Other components that can be added to blocking buffers include, for example, salts, metal-chelating reagents, non-specific binding reagents, non-denaturing zwitterionic detergents, and the like.

The methods of the present invention relate in part to the discovery that the use of a non-human mammalian globulin such as BGG in the blocking buffer of an assay system for detecting a human or humanized antibody in non-human serum results in significantly decreased background signal and variation.

Accordingly, an embodiment of the invention includes an assay system for detecting a human target molecule in a non-human biological matrix, wherein a blocking agent comprises a non-human mammalian globulin. The non-human mammalian globulin can be, for example, bovine gamma globulin (BGG). Other non-human mammalian globulins can also be used in blocking buffer. These include, but are not limited to, mouse IgG, rabbit IgG, donkey IgG, and the like. The blocking buffer may additionally comprise conventional blocking agents, such as bovine serum albumin, gelatin, egg albumin, casein, non-fat milk, and the like. The assay methods of the invention are exemplified herein with embodiments where the target molecule is a humanized antibody. It is understood, however, that the described embodiments can also be used to detect non-human target molecules, for example where the non-human (first species) molecule is disposed in a different (second species) biological matrix. For detecting a non-human molecule in a second species biological matrix, a globulin molecule of a species other than that of the second species can be used.

In one embodiment, non-human mammalian globulin, for example, BGG, is present as a component of the blocking buffer. The non-human mammalian globulin can be lacking in the sample buffer and/or in the detection buffer. In an embodiment utilizing a bridging ELISA format, the non-human globulin can be present only in the blocking buffer and not in the sample buffer or detection buffer. In a direct ELISA format, the non-human globulin can be present in the blocking buffer and in the sample and detection agent buffers.

D. Suitable Assays

In the methods of the invention, once the capture reagent has been blocked with a blocking buffer containing a mammalian globulin and the blocked capture reagent is further reacted with a sample containing the molecule of interest to capture the target molecule, the captured target molecule is detected and/or quantitated, for example, with a detection agent that generates a detectable signal.

Assay systems utilizing a detection agent to quantitate captured molecules including antibodies are well known. Examples of immunoassays useful in the invention include, but are not limited to, radioimmunoassay (RIA), fluoroluminescence (FLA), chemiluminescence assay (CA), enzyme-linked immunosorbant assay (ELISA) and the like. See, for example, Johnstone and Thorpe, 1996, In: Blackwell, Immunochemistry in Practice, 3rd ed. (Blackwell Publishing, Malden, Mass.); Ausbul et al., eds., 2003, Current Protocols in Molecular Biology, Wiley & Sons (Hoboken, N.J.); Ghindilis et al., eds., 2003, Immunoassay Methods and Protocols, (Blackwell Publishing, Malden, Mass.); and U.S. Patent Publication No. 20030044865. The immunoassay can be a solid phase assay, a liquid phase assay, and the like.

1. ELISA

Immunoassay systems include, for example, solid-phase ELISA and capture ELISA. In capture ELISA, immobilization of the target molecule to a solid phase can be accomplished by known methods. The target molecule can be immobilized, for example, by insolubilizing a capture reagent before the assay procedure, such as by adsorption of the capture reagent to a water-insoluble matrix or surface (See U.S. Pat. No. 3,720,760). The capture reagent can also be insolubilized by non-covalent or covalent coupling to a water-insoluble matrix or surface, for example, using glutaraldehyde or carbodiimide cross-linking, with or without prior activation of the assay surface with, for example, nitric acid and a reducing agent. See, for example U.S. Pat. No. 3,645,852 and Rotmans et al., 1983, J. Immunol. Methods, 57:87-98. The target molecule can also be immobilized after the assay procedure, for example, by immunoprecipitation.

The capture reagent can be, for example, an antibody or a mixture of different antibodies that binds a target antigen. The capture reagent can be an antibody/antigen complex, where the antigen of the complex is available to bind a target molecule in a sample. In a further embodiment, the capture reagent can be an anti-isotype specific antibody complexed to an antibody that specifically binds a therapeutic antibody. For example, the capture reagent may be a goat anti-human IgG Fc specific antibody complexed to an anti-therapeutic IgG monoclonal antibody. In one embodiment, the anti-therapeutic IgG monoclonal antibody is an anti-2H7 antibody.

a) Solid Phase

The capture reagent can be immobilized on a solid phase for use in a solid phase ELISA assay. Any inert support or carrier that is essentially water insoluble and useful in immunoassays, including supports in the form of, e.g., surfaces, particles, porous matrices, sensor chips, and the like can be used as the solid phase or assay surface. Examples of commonly used supports include small sheets, Sephadex, polyvinyl chloride, plastic beads, microparticles, assay plates, or test tubes manufactured from polyethylene, polypropylene, polystyrene, and the like. Such supports include 96-well microtiter plates, and biosensor chips such as BiaCore Sensor chips, as well as particulate materials such as filter paper, agarose, cross-linked dextran, and other polysaccharides. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are suitably employed for capture reagent immobilization. In an embodiment, the immobilized capture reagent is coated on a microtiter plate. A solid phase such as a multi-well microtiter plate can be used to analyze several samples at one time.

The solid phase can be coated with the capture reagent that may be linked by a non-covalent or covalent interaction or physical linkage, as desired. Techniques for attachment include those described in U.S. Pat. No. 4,376,110 and the references cited therein. If covalent attachment of the capture reagent to the assay surface is utilized, the plate or other solid phase may be incubated with a cross-linking agent together with the capture reagent. Commonly used cross-linking agents for attaching the capture reagent to a solid phase substrate include, for example, 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates capable of forming cross-links in the presence of light.

If polystyrene or polypropylene plates are utilized, the wells in the plate can be coated with the capture reagent (typically diluted in a buffer such as 0.05 M sodium carbonate) by incubation for at least about 10 hours, for example, at least overnight, at temperatures of about 4-20° C., such as about 4-8° C., and at a pH of about 8-12, such as about 9-10, or about 9.6. If shorter coating times (1-2 hours) are desired, the plate can be coated at 37° C., or the plates can comprise nitrocellulose filter bottoms such, as for example, Millipore MULTISCREEN™. The plates may be stacked and coated in advance of the assay, allowing for an immunoassay to be carried out simultaneously on several samples in a manual, semi-automatic, or automatic fashion, such as by using robotics.

b) Blocking

The coated assay surface (solid phase), for example, microtiter plates, are typically treated with a blocking agent that binds and saturates non-specific binding sites to prevent unwanted binding of free ligand to excess binding sites of the solid phase. A non-human mammalian globulin can be used as the blocking agent of the blocking buffer, for example, in an assay for detecting a human target molecule such as a human or humanized antibody. The blocking treatment typically takes place under conditions of ambient temperatures and for a time period of about 1-4 hours, for example about 1.5 to 3 hours, or about 2 hours.

c) Sample Addition

After coating and blocking, the sample to be analyzed (for example, serum), can be diluted, for example, by about 10% by volume. Buffers that may be used for dilution include, for example,

(a) phosphate buffered saline (PBS) containing 0.5% BSA, 0.05% TWEEN 20™ detergent (P20), 5 mM EDTA, 0.25% Chaps surfactant, 0.35M NaCl, and 0.05% Proclin-300, pH 8.01;

(b) PBS containing 0.5% BSA, 0.05% Proclin-300, and 0.05% P20, pH 7.3;

(c) PBS containing 0.5% BSA, 0.05% P20, 0.05% Proclin-300, 5 mM EDTA, and 0.35 M NaCl, pH 6.39;

(d) PBS containing 0.5% BSA, 0.05% P20, 0.05% Proclin-300, 5 mM EDTA, 0.2% beta-gamma globulin, 0.25% CHAPS, and 0.35 M NaCl; and

(f) PBS containing 2% BSA, 0.05% Proclin-300, and 0.05% P20, pH 7.3;

(g) PBS containing 0.5% BSA, 0.05% Proclin-300, and 0.05% P20, pH 7.3, 0.1% Triton X-100;

(h) PBS containing 0.5% BSA, 0.05% Proclin-300, and 0.05% P20, pH 7.3, 0.1% Tween-80;

(i) PBS containing 0.5% BSA, 0.05% Proclin-300, and 0.05% P20, pH 7.3, 0.1% n-octyl-b-D-glucopyranoside.

For sufficient sensitivity, the immobilized capture reagent is generally in molar excess of the maximum molar concentration of the target molecule anticipated in the sample after appropriate dilution. Depending on the target molecule, the capture reagent may compete for binding sites with the detecting antibody, yielding inaccurate results. Therefore, the final concentration of the capture reagent will normally be determined empirically to maximize the sensitivity of the assay over the range of interest.

In some embodiments, the sample buffer may include additional ingredients. For example, mammalian globulin such as BGG can be added to the sample buffer in some embodiments.

d) Incubation

Conditions for incubation of sample and capture reagent are selected to maximize sensitivity of the assay, and to minimize dissociation. Incubation time depends primarily on temperature, with time of incubation generally decreasing with increasing temperature. The incubation time can be, for example, overnight and can be, for example at room temperature. Increasing the temperature, for example, to about 30° C. can reduce the incubation time, for example, to about 1-3 hours. Decreasing the incubation temperature, for example, to about 4° C. can increase the incubation time, for example, to about 24-48 hours. If the sample is a biological fluid, incubation times may be lengthened by adding a protease inhibitor to the sample to prevent proteases in the biological fluid from degrading the analyte.

The pH of the incubation buffer is chosen to maintain a significant level of specific binding of the capture reagent to the analyte being captured. The pH of the incubation buffer is generally about 6-9.5, for example about 7-8. Various buffers may be employed to achieve and maintain the desired pH during this step, including borate, phosphate, carbonate, Tris-HCI or Tris-phosphate, acetate, barbital, and the like. The particular buffer employed is usually not critical; however, in individual assays one buffer may be preferred over another.

e) Wash

The sample can be separated from the immobilized capture reagent with a wash solution to remove uncaptured analyte from the system. The wash solution is generally a buffer, and can be one of the incubation buffers described above, for example. The pH of the wash solution is determined as described above for the incubation buffer, and can be about 6-9, for example about 7-8. Washes may be done one or more times. Minimizing the number of washes, however, can help to retain molecules that bind the target molecule with low affinity; however, minimizing washes can increase the background of the assay. In one embodiment, three washes are used. The temperature of the wash solution is typically from about 0-40° C., and can be about 4-30° C. An automated plate washer may be utilized. A cross-linking agent or other suitable agent may be added to the wash solution to covalently attach the captured analyte to the capture reagent.

f) Detection

Following removal of uncaptured target molecules from the system, for example by washing, the captured target molecules can be contacted with a detecting agent that binds and enables detection of the captured target molecule, such as captured antibody, for example at room temperature. When the target molecule is a humanized therapeutic antibody, the detecting agent can be, for example, an anti-isotype antibody of a different species. If the therapeutic antibodies are human IgG, for example, the detecting agent may be a murine anti-human IgG antibody. In an embodiment, the target molecule is murine monoclonal antibody and the detecting agent is sheep anti-mouse IgG.

The temperature and time for contacting the target molecule with the detecting agent is dependent primarily on the detection means employed. For example, when horseradish peroxidase (HRP) conjugated to sheep anti-mouse IgG is used as the means for detection, the detecting agent can be incubated with the captured target molecule for about 0.5-2 hours, for example, about 1 hour. The system can be washed as described above to remove unbound detecting agent from the system, and developed by adding peroxidase substrate and incubating the plate for about 5 minutes at room temperature, or until good color is visible.

A molar excess of the detecting agent is typically added to the system after the unbound target molecule has been washed from the system. The detection agent may be a polyclonal or monoclonal antibody, and can be, for example, a monoclonal antibody, such as a murine monoclonal antibody. The detecting agent may be directly or indirectly detectable. If the detecting agent is an antibody that is not directly detectable, for example, not labeled, the detecting antibody can be detected by addition of a molar excess of a second, labeled antibody directed against the isotype and animal species of the detecting antibody.

g) Affinity

The affinity of the detecting agent must be sufficiently high such that small amounts of target molecule can be detected. A fluorimetric or chemilimunescent label moiety has greater sensitivity in immunoassays as compared to a conventional colorimetric label. The binding affinity of the selected detecting agent must be considered in view of the binding affinity of the capture reagent, such that the detecting agent does not strip the target molecule from the capture reagent.

h) Label

The label moiety can be any detectable functionality that does not interfere with the binding of the captured target molecule to the detecting agent. Examples of suitable label moieties include moieties that may be detected directly, such as fluorochrome, chemiluminscent, and radioactive labels, as well as moieties, such as enzymes, that must be reacted or derivatized to be detected. Examples of such labels include the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e g., firefly luciferase bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HPP, lactoperoxidase, or microperoxidase, biotin/avidin, biotin/streptavidin, biotin/Streptavidin-β-galactosidase with MUG, spin labels, bacteriophage labels, stable free radicals, and the like.

Conjugation of the label moiety to the detecting agent, such as for example an antibody, is a standard manipulative procedure in immunoassay techniques. See, for example, O'Sullivan et al., 1981, “Methods for the Preparation of Enzyme-antibody Conjugates for Use in Enzyme Immunoassay,” in Methods in Enzymology, Langone and Van Vunakis, Eds., Vol. 73 (Academic Press, New York, N.Y.), pp. 147-166. Conventional methods are available to bind the label moiety covalently to proteins or polypeptides. For example, coupling agents such as dialdehydes, carbodiimides, dimaleimides, bis-imidates, bis-diazotized benzidine, and the like, may be used to label antibodies with the above-described fluorescent, chemiluminescent, and enzyme labels. See, for example, U.S. Pat. No. 3,940,475 (fluorimetry) and U.S. Pat. No. 3,645,090 (enzymes); Hunter et al., 1962, Nature, 144:945; David et al., 1974, Biochemistry, 13:1014-1021; Pain et al., 1981, J. Immunol Methods, 40:219-230; and Nygren J., 1982, Histochem. and Cytochem., 30:407-412. Fluorescent or chemiluminescent labels can be used to increase amplification and sensitivity to about 5-10 pg/ml.

The amount of target molecule bound to the capture reagent can be determined by washing away unbound detecting agent from the immobilized phase, and measuring the amount of detecting agent bound to the target molecule using a detection method appropriate to the label. The label moiety can be, for example, an enzyme. In the case of enzyme moieties, the amount of developed signal, for example, color, is a direct measurement of the amount of captured target molecule. For example, when HRP is the label moiety, color is detected by quantifying the optical density (O.D.) at 650 nm absorbance. In another embodiment, the quantity of target molecule bound to the capture reagent can be determined indirectly. The signal of an unlabeled detecting agent may be amplified for detection with an anti-detecting agent antibody conjugated to a label moiety. For example, the signal of an unlabeled mouse antibody that binds the target molecule may be amplified with a sheep anti-mouse IgG antibody labeled with HRP. The label moiety is detected using a detection method appropriate to the label. For example, HRP may be detected by reacting HRP with a colorimetric substrate and measuring the optical density of the reacted substrate at 650 nm absorbance.

2) Bridging Format ELISA

In a conventional, direct ELISA system, the capture reagent and detection agent differ from each other in structure and/or species. For example, the capture reagent can be sheep anti-human IgG, and the detection reagent can comprise goat anti-human IgG.

In a Bridging ELISA system, the capture reagent and detection agent are similar in structure and/or species, and can be derived from the same polyclonal antibody. Bridging ELISA's have been observed to exhibit a reduced non-specific background and variation of background in various assays. In one embodiment of the invention, both the capture reagent and detection agent comprise the same polyclonal antibody, and can be or comprise, for example, sheep anti-human IgG.

E. Assays Using Non-Mammalian Gamma Globulin

A target-specific binding molecule is typically needed to assay a sample in a biological matrix where high sensitivity is needed. Target specific molecules usually help reduce both assay background and individual background variation, and therefore provide a high assay sensitivity. While both polyclonal and monoclonal anti-rhuMAb2H7 idiotypic molecules were under development, during the present investigations, efforts were first focused on the development of an assay for rhuMAb2H7 using a CD20 molecule that could be specifically recognized by rhuMAb2H7 with high affinity. These efforts failed to result in a highly sensitive and accurate assay for rhuMAb2H7.

The studies described in the Examples below, however, resulted in the discovery of an ELISA assay able to quantify antibodies such as rhuMAb2H7 in non-human serum with high sensitivity and accuracy, and independent of any target-specific molecules. The methods disclosed herein provide a general method for the quantification of analytes of a first species in the biological matrix of a second species, particularly closely related species. In one embodiment, the methods provide for detection and quantitation of molecules of a first species such as human antibodies, humanized antibodies, chimeric antibodies, and the like, in a biological matrix of a second species, for example, in the serum of a non-human species such as cynomolgus monkey body fluid.

The assay methods disclosed herein do not require use of a capture reagent that is specific for the target molecule to be detected and/or quantified (i.e., a target-specific molecule), and may be used to quantitate a wide variety of analytes, including human antibodies, human chimeric antibodies, humanized antibodies, fragments thereof, and the like. These methods have a wide application, for example, in humanized antibody pharmacokinetic assay development, especially in the early stage of drug development where animal studies are essential, but reagents are not readily available.

When a proper target specific molecule is unavailable, as in the case of CD20 for detecting the 2H7 therapeutic antibody described herein, efforts can focus on removing potentially interfering components as much as possible. Pre-adsorption of capture reagent with potentially interfering material can greatly reduce the background compared to a capture reagent without pre-adsorption, for example, background due to the presence of serum proteins. In the Examples below, the capture reagent used, sheep anti-human IgG (H+L), was monkey serum adsorbed. This resulted in a relatively low background compared to other capture reagents. However, despite the fact that cynomolgus monkey IgG has strong similarity to human IgG and is likely a major contributor to the interference, simply removing cynomolgus monkey IgG binding components in the capture reagent was found insufficient. Further adsorption of the capture reagent with cynomolgus monkey IgGs purified from high background individuals failed to result in a further background reduction, suggesting that serum proteins other than IgGs also interfered with the assay. The high variation of the background noted among individual cynomolgus monkey serum was also likely a result of interference from serum proteins other than IgG itself. These components may have different concentrations and/or affinities to the capture reagent, thus giving a high background variation.

While it is not realistic to find and remove all interfering components present in the serum, maximizing the background to diminish the background variation was investigated as a strategy of an immunoassay system. Increasing serum concentration is one way to maximize background and lower background variation, however, a significantly increased background also reduces the sensitivity of the assay.

As disclosed herein, it was discovered that addition of non-human gamma globulin (BGG) to blocking buffer dramatically reduces background variation with only a relatively small increase in serum background. Without limitation by theory, it is believed that BGG interacted weakly with the capture reagent (e.g. sheep anti-human IgG (H+L)), and caused a general increase in the background as evidenced in the assay blank. The interaction was too weak to abolish the human IgG binding completely, however, or to cause a substantially high background with cynomolgus monkey serum.

The addition of BGG in the sample and/or detection agent diluent buffers, however, was found to interfere with human antibody detection, e.g., rhuMAb2H7 detection, by causing a reduction in the signal. The interaction of BGG with the capture reagent masks some of the binding sites of the capture reagent and potentially interacts with the detection agent as well, and therefore yielded a decrease in the absolute rhuMAb2H7 signal. On the other hand, the interaction between BGG and the capture reagent was somewhat stronger than that from the cynomolgus monkey serum proteins, and therefore resulted in a reduced background variation. Several factors may potentially contribute to the stronger interaction of the human antibody, e.g., rhuMAb2H7, with BGG, such as a relatively high concentration of BGG in the buffer, a higher affinity of BGG to the human antibody, e.g., rhuMAb2H7, or a combination of these factors.

Removing BGG from both the sample and detection agent buffers was found to enhance the absolute human antibody signal, e.g., rhuMAb2H7 signal, and further improve the signal-to-noise ratio. This observation is consistent with the hypothesis that BGG masks some binding sites of both the capture reagent and the detection agent for the human antibody, e.g., rhuMAb2H7. Therefore, while using BGG in the blocking step results in a lower background variation, removing BGG from the buffers results in a higher signal-to-noise ratio.

A bridging format that used the same reagent (e.g. sheep anti-human IgG) for both the capture reagent and the detection agent was found to produce a lower background and smaller individual serum background variation than a conventional, direct ELISA format. Several potential explanations can be raised. First, since the detection agent sheep anti-human IgG was also monkey serum adsorbed, the chances of binding to a non-specific protein captured during the sample incubation were reduced. Secondly, in a bridging format where the detection agent was derived from the capture reagent, an analyte that contains two identical binding sites was preferentially detected. Cynomolgus serum proteins other than IgGs also contribute to the background as suggested by the pre-adsorption experiment. These serum proteins, however, cannot effectively serve as a bridge when both the capture reagent and the detection agent are derived from the same molecule, and therefore, their recognition in the bridging format will be minimized, resulting in a further decrease in the serum background.

As shown in the Examples below, a cynomolgus monkey serum rhuMAb2H7 PK assay with a high sensitivity was developed. Buffers without the addition of pooled cynomolgus monkey serum were used successfully in the sample and detection agent buffers, and a standard curve was generated in buffer as well. The practice reduces the cost and simplifies preparation of the assay for automation, and further minimizes potential risks in obtaining/maintaining comparable reagents to ensure reproducible results. The assay is highly sensitive, using a minimum serum dilution of 1:10. The assay is also very robust, with small intra- and inter-assay variability. For the rhuMAb2H7 molecule, the assay revealed the pharmacokinetics of the molecule in several cynomolgus monkey studies, as well as in studies in rodent species.

The assay of the present invention is independent of any specific target molecule, and may be used to quantify a variety of molecules, including human/humanized IgGs. Optimization important to development of the rhuMAb2H7 assay disclosed herein can be applied to assays for other molecules, including other human/humanized IgGs, as shown in the following examples. The assay has a general application and has been used, for example, to measure rhuMAb2H7 in monkey, rat, and mouse serum (See Table 1).

TABLE 1

| | Spike Recovery of 100 ng/ml |
|---|---|
| Species | rhuMAb2H7 in 10% serum |
| Rat | 97% |
| Mouse | 99% |

Results of studies performed in rat and mouse are shown in Table 1. The data shows high rates of spike recovery from these two species using an optimized generic (non-target specific) assay of the invention. Moreover, preliminary results also suggested that rhuMAb2H7 can be detected in the serum of other non-human primates such as baboon, rhesus, and African green monkeys (See Table 2).

TABLE 2

| Species | Recovery of 100 ng/ml of rhuMAb 2H7 in 10% serum |
|---|---|
| Rhesus monkey | 93% |
| African green | 85% |
| Baboon | 81% |

The data, as shown in Table 2, shows recoveries ranging from 81-93% across these three species.

The following examples are offered by way of illustration and not by way of limitation. The disclosures of all citations in the specification are expressly incorporated herein by reference.

### EXAMPLE 1

### Preadsorption to Reduce Background/Variation

1. Preadsorption to Remove Cynomolgus Monkey IgG Cross-Reactivity

Various capture reagents were screened for use in methods disclosed in the present invention. Cynomolgus monkey serum-adsorbed sheep anti-human IgG heavy (H) and light (L) chain (Cat #CUS1684) is commercially available from The Binding Site (San Diego, Calif.), and showed promising potential. As discussed below and shown in Table 3, however, use of this capture reagent resulted in high and variable background. Therefore, in an attempt to reduce the background and background variation, monkey serum-adsorbed sheep anti-human IgG (H+L) was further adsorbed with purified cynomolgus monkey IgGs obtained from serum of problematic individual monkeys (high background) to further remove any IgG-binding components in the capture reagent.

Cynomolgus monkey IgGs were first purified by a HiTrap Protein G column (Pharmacia) following the procedures recommended by the manufacturer. Briefly, the column was washed with water and equilibrated with 20 mM sodium phosphate, pH 7.0. About 1 ml of individual cynomolgus monkey serum that gave a high background during the initial screening was injected onto the column with a syringe. The column was then washed with five column volumes of the 20 mM sodium phosphate buffer and eluted with 0.1 M glycine, pH 2.7. The eluent was dialyzed against PBS overnight at 4° C., and the concentration was measured by absorbance at 280 nm using an estimated extinction coefficient of 1.36.

The purified cynomolgus IgG was then coupled to controlled pore-glass (CPG) beads using a standard procedure. Briefly, the beads were first allowed to swell in a snap-cap tube containing distilled water. The supernatant was removed by vacuum suction and discarded. Freshly prepared 1% sodium metaperiodate was added to the tube in a volume equal to that of the wet beads, and the suspension was rotated gently at room temperature for 30 minutes. After the beads settled, the supernatant was decanted and the beads were washed with PBS five times to remove excess periodate. The purified cynomolgus monkey IgG was added to the activated beads, and the suspension was mixed thoroughly before the beads were allowed to settle. Two milligrams of solid sodium cyanoborohydride was added, and the mixture was mixed at 4° C. for 40 hours. The coupled resin was washed in PBS several times before being blocked with 1 M ethanolamine at pH 8.0 overnight. The resin was then washed with and stored in PBS.

The capture reagent was then preadsorbed with the purified cynomolgus monkey IgG according to the following procedure. The capture reagent (100 μl of 1 μg/ml in sodium carbonate (pH 9.6)) was added to wells of a 96-well microtitre plate and the plate was incubated at 4° C. overnight to immobilize the capture reagent. The plate was then washed three times with PBS containing 0.05% polysorbate-20. The immobilized capture reagent was adsorbed against pooled cynomolgus monkey IgG sera, or against a combination of pooled cynomolgus monkey sera plus sera from one or two individual monkeys that tended to produce high backgrounds, at 1% or 10% serum concentrations, as shown in Table 3.

Buffer A (PBS containing 0.5% BSA, 0.05% Tween-20, and 0.05% Proclin-300) was added to washed plates and incubated at room temperature for about 2 hours to block the plate. The plates were washed 3 times and blotted dry. Buffer A was also used to dilute both samples and the detection agent.

2. Results

TABLE 3

| | Capture reagent | | | Mean | |
|---|---|---|---|---|---|
| | ADDITIONAL | Serum | Number of | Background | CV % of |
| Run | pre-treatment^{a} | Concentration | Individuals | (O.D. 450-650 nm) | Background |
| 1 | none | 1% | 10 | 0.181 | 85 |
| 2 | none | 10% | 8 | 0.308 | 41 |
| 3 | monkey serum | 10% | 8 | 0.314 | 42 |
| | adsorption plus | | | | |
| | pooled cynomolgus | | | | |
| | monkey IgG | | | | |
| | fractions | | | | |
| 4 | monkey serum | 10% | 8 | 0.299 | 46 |
| | adsorption plus | | | | |
| | one problematic | | | | |
| | individual and | | | | |
| | pooled | | | | |
| | fractions | | | | |
| 5 | monkey serum | 10% | 8 | 0.261 | 47 |
| | adsorption plus | | | | |
| | two problematic | | | | |
| | individual and | | | | |
| | pooled | | | | |
| | fractions | | | | |

^{a}The capture reagent was sheep anti-human IgG heavy (H) and light (L) chain (Cat #CUS1684) obtained from The Binding Site (San Diego, CA). This capture reagent as available commercially is already preadsorbed with cynomolgus monkey serum. Thus, the pre-treatments listed in this column refer to additional pre-treatment adsorption steps.

As shown in Table 3, preadsorption of the capture reagent with 1% cynomolgus monkey serum resulted in a mean background of 0.181 O.D., much lower than with 10% serum (0.308 O.D.). Background variation using the 1% serum, however, was extremely high, with a CV % of 85. See Table 3.

The lower variation observed with the 10% serum suggests a background maximizing effect with a higher concentration of the serum. The background from cynomolgus monkey serum likely resulted from non-specific protein binding to the capture reagent. Present at different concentrations in individual cynomolgus monkey serum, these non-specific proteins contributed to the variation of individual serum backgrounds. Further dilution of the serum may reduce background variation and mean serum background, but would result in an assay not sensitive enough for PK analysis.

In contrast, data shown in Table 3 indicated that increasing the concentration of the serum resulted in suppression of the background variation. As the percentage of serum used in the assay is increased, the total amount of non-specific interacting proteins captured on the plate will increase until a maximum amount is reached, when the capture reagent becomes a limiting reagent. Since non-specific interactions are usually weak, the maximized signal may be low enough to produce a workable assay.

With 10% cynomolgus monkey serum, however, individual background variation was still very high, even though it was greatly lower than that of 1% serum. Using a serum concentration above 10% may suppress variation further, but is likely to result in an even higher background that itself may pose another challenge in assay development.

Overall, pre-adsorbing the capture reagent antibody with cynomolgus monkey IgG to further remove any IgG binding components in the capture reagent did not result in any appreciable improvement in minimizing variation of individual cynomolgus monkey serum background. This observation suggested that, with the current format, variation of the individual serum backgrounds resulted from proteins other than the cynomolgus monkey immunoglobulins.

### EXAMPLE 2

### BGG (Bovine Gamma Globulin) in Blocking Buffer

Use of BGG in blocking buffer, sample diluent, and detection agent diluent was analyzed to determine if background levels and background variation could be improved. Sheep anti-human IgG heavy (H) and light (L) chain capture reagent (Cat #CUS1684) and sheep anti-human IgG (H+L) HRP conjugate detection reagent (Cat #CUS1684.H) were purchased from The Binding Site (San Diego, Calif.). Humanized mAbs can be generated by known methods. rhuMAb2H7 was generated as described in WO 04/056312. Herceptin®, Xolair®, Avastin™ and Raptiva® were generated according to the procedures described in Carter et al., PNAS 89: 4285-4289 (1992), U.S. Pat. No. 6,172,213, Presta et al., Cancer Research 57:4593-4599 (1997), and U.S. Pat. No. 6,037,454, respectively. Goat anti-human IgG (H+L) horseradish peroxidase (HRP) (detection agent) was purchased from American Qualex (San Clemente, Calif.). Individual cynomolgus sera were obtained from BiochemMed (VA). Maxisorp Nunc-immuno 96-well microtiter plates were purchased from Nalge Nunc International (Rochester, N.Y.). The HRP detection agent substrate 3,3′,5,5′-tetramethylbenzidine (TMB) and H₂O₂ were obtained from KPL (Gaithersburg, Mass.). Bovine serum albumin (bovuminar Cohn Fraction V, pH7) was obtained from Serologicals Corp (cat#3322-90, Ontario, Canada) and Proclin 300 was from Supelco (Bellefonte, Pa.). A 20× solution of phosphate buffered saline (PBS) containing 1% polysorbate 20 was purchased from MediaTech Cellgro (Herndon, Va.). Both bovine γ-immunoglobulin (BGG) and 3-[(3 cholamidopropyl)dimethylammonio]-1-propane-sulfonate (CHAPS) were from Sigma (St. Louis, Mo.). An EL 404 microplate autowasher from Bio-Tek Instruments, Inc. (Winooski, Vt.) was used for all the washing steps in ELISA. A Spectra Max250 plate reader (Molecular Devices Corporation, Sunnyvale, Calif.) was used to record signals in ELISA using absorbance at 450 nm subtracted from that at 650 nm.

1. Format for ELISA: Adsorption of Sheep Anti-Human IgG(Heavy+Light Chain) to Remove Cynomolgus Monkey IgG Cross-Reactivity.

a. Application of the Capture Reagent to the Assay Surface and Preadsorption of the Capture Reagent.

A volume of 100 μl of capture reagent (1 μg/ml sheep anti-human IgG (H+L) in sodium carbonate (pH 9.6) was added to a 96-well microtiter plate and the plate was incubated at 4° C. overnight. The plate was then washed 3 times with washing buffer (PBS with 0.05% polysorbate-20).

b. Application of Sample to the Capture Reagent.

Blocking buffer (200 μl, PBS/0.5% BSA/0.05% P20, 0.05% Proclin 300, 0.25% CHAPS, 0.2% BGG, 5 mM EDTA, 0.35 M NaCl, pH 8.0) was added to washed, pre-absorbed capture reagent. The plate was sealed and incubated at room temperature for 2 hours with gentle agitation. After washing three times and blotting dry, 100 μl of rhuMAb2H7 standard, controls, serum blanks, and serum samples in buffer (having the same composition as the blocking buffer) were added to the plate. The plate was subsequently sealed with a plate sealer. After incubating at room temperature for another hour with a gentle agitation, the plate was washed six times with washing buffer and blotted dry.

c. Quantification of Sample Analyte with Detection Agent

Diluted detection agent (100 μl) was added and the plate was sealed and incubated at room temperature for one hour with a gentle agitation. Then the plate was washed another six times and blotted dry before the addition of 100 μl of an equal volume of TMB and H₂O₂. After incubating at room temperature for 15 minutes without agitation, the reaction was stopped by adding another 100 μl of 1 M H₃PO₄. Absorbance at 450 nm was subtracted from that at 650 nm, was read from a Spectra Max250 plate reader (Molecular Devices Corporation, Calif.), and the data was processed using SoftmaxPro software provided by the manufacturer.

2. Use of BGG in All Buffers (Blocking Buffer, Sample Buffer, and Detection Buffers)

In a further attempt to minimize the assay background while establishing better control over the background variation, several buffer solutions (buffers A-E as shown in Table 4) containing various buffer additives, including BGG (bovine gamma globulin), were prepared and used in the blocking buffer, sample buffer, and detection agent buffer. These buffers were then screened in the ELISA assay as described above to ascertain their efficacy in reducing the background and variation. The data are shown in Table 4.

TABLE 4

| | Buffer | | | Assay | | |
|---|---|---|---|---|---|---|
| Buffer | Additives | pH | Signal³ | Blank⁴ | Mean | CV %⁶ |
| A¹ | none | 7.30 | 2.041 | 0.012 | 0.083 | 47 |
| B¹ | 5 mM EDTA | 6.39 | 1.911 | 0.007 | 0.058 | 62 |
| | 0.35 M NaCl | | | | | |
| C¹ | 5 mM EDTA | 8.01 | 1.878 | 0.113 | 0.142 | 22 |
| | 0.35 M NaCl | | | | | |
| | 0.25% CHAPS | | | | | |
| | 0.2% BGG | | | | | |
| D¹ | 5 mM EDTA | 8.98 | 1.724 | 0.142 | 0.165 | 18 |
| | 0.35 M NaCl | | | | | |
| | 0.25% CHAPS | | | | | |
| | 0.2% BGG | | | | | |
| E² | none | | 2.098 | 0.009 | 0.202 | 0.202 |

¹Basic buffer components were PBS/0.5% BSA, 0.05% Tween-20, and 0.05% Proclin-300.
²Basic buffer components were 55 mM HEPES/0.5% BSA, 25 mM HEPES sodium salt, 2% Triton X-100, and 0.05% Proclin-300.
³O.D. measurement at 450-650 nm at a rhuMAb2H7 concentration of 240 ng/ml, n = 8.
⁴O.D. measurement at 450-650 nm. n = 8.
⁵Mean serum background measured at 450-650 nm.
⁶CV % of the background.

Table 4 lists the components of each buffer. Buffer A was prepared as a standard assay buffer, and contained 0.5% BSA, 0.05% Tween-20, and 0.05% Proclin-300. Buffers C and D were prepared with the same components as Buffer A, but with the buffer additives listed in Table 4, including BGG. N=8 for all experiments.

Buffers were tested in the ELISA assay described above. The buffers were used to block the plate after coating of the capture reagent (sheep anti-human IgG (H+L), monkey serum adsorbed), and to dilute both the samples (10% individual cynomolgus monkey serum and rhuMAb2H7), and the detection agent (goat anti-human IgG•HRP). The assay blank listed in Table 4 refers to a buffer sample containing no serum or rhuMAb2H7.

3. Results

Comparisons of the rhuMAb2H7 signal and the assay background for both the assay blank and 10% cynomolgus monkey serum were made under different buffer conditions using ELISA.

Among the buffers screened, only buffer B lowered both the average serum background and the background from the assay blank compared to the regular assay diluent A (Table 4). Variation among individual cynomolgus monkey sera, however, was 62%, a higher variation than seen when using the standard assay diluent A (47%).

Buffer E was also found to lower background compared to the assay blank. This buffer appeared to enhance non-specific interactions between cynomolgus monkey serum and the capture reagent/detection reagent, as indicated by the observed increase in mean serum background. This increase was similar for all individual cynomolgus monkeys in the experiment, since the observed variation of individual serum background was similar to that of buffer A (Table 4).

Buffers C and D comprised the same buffer compositions, but had different pH values (8.98 and 8.01 respectively). Both buffers C and D caused an increase in the background of the assay blank, indicating that one or more buffer components were weakly interacting with both the capture reagent and the detection agent. Since these buffers comprise additional additives, it was not surprising that the background of the mean cynomolgus monkey serum increased.

Variation among individual cynomolgus monkey sera dropped significantly when buffers C and D were used, by 22% and 18% respectively (Table 4). This decrease likely resulted from additional interactions between the buffer additives and the capture reagent/detection agent, since the interaction maximized the background, masking the differences in individual monkey sera to produce a decreased background variation.

Also, as seen in Table 4, the difference between the background and the assay blank of each of buffers C and D narrowed significantly. This narrow difference between the background and the assay blank, combined with the observation of reduced serum background variation obtained from use of these buffers, suggested that with buffers C and D, non-specific interactions from cynomolgus monkey serum to the capture reagent/detection agent became a minor contributor to the background of the serum, compared to the contribution of the additional components present in these buffers.

The change in major background contributors when buffers C and D were used caused a decreased variation among individual sera, as well as a close similarity of the serum background to that of the assay blank. This result made it possible to use the assay buffer alone to dilute a standard curve and sample, eliminating the need to find a suitable and representative pooled cynomolgus monkey serum for addition to the buffer.

It was also discovered that the higher pH of buffer D (8.98) compared to buffer C (8.01) caused a further reduction in the individual cynomolgus monkey serum variation (Table 4).

Use of buffers C and D also reduced the rhuMAb2H7 signal (Table 4). A possible explanation for this observation is that the interaction of the additional additives in these buffers to the detection agent effectively masked detection of rhuMAb2H7, resulting in a decreased signal. Due to similarities between the capture reagent and the detection agent (anti-human IgGs from two different species), it is not surprising that the additional additives in buffers C and D can interact with both reagents.

4. Optimization of Buffer D to Restore rhuMAb2H7 Signal

In an attempt to restore the rhuMAb2H7 signal, a systematic exploration of the contribution of each additive in buffer D was undertaken to isolate parameters that caused the decreased signal.

Buffer D contained four additional additives not present in Buffer A. Buffers A1-A4 were prepared, each having one additional additive from Buffer D that was not present in the original Buffer A. The effects of the additional additives on the signal-to-noise ratio in the detection agent dilution step were measured. Sheep anti-human IgG (H+L), (monkey serum adsorbed) was used as a capture reagent. The detection agent was goat anti-human IgG (H+L)●HRP. The buffer compositions were as described for Table 4. Results are shown in Table 5.

TABLE 5

| | | | 10% cynomolgus | |
|---|---|---|---|---|
| | Conjugate Dilution | 240 ng/ml 2H7 | monkey serum | Signal-to- |
| Name | Buffer | (O.D. 450-650 nm) | (O.D. 450-650 nm) | Noise Ratio |
| A | Buffer A | 2.229 | 0.114 | 19.6 |
| A1 | Buffer A + 0.35 M NaCl | 2.002 | 0.088 | 22.8 |
| A2 | Buffer A + 0.2% BGG | 2.280 | 0.147 | 15.6 |
| A3 | Buffer A + 5 mM EDTA | 2.130 | 0.097 | 22.1 |
| A4 | Buffer A + 0.25% | 2.146 | 0.100 | 21.6 |
| | CHAPS | | | |
| D | Buffer D | 0.962 | 0.044 | 22.1 |
| D1 | Buffer D - BGG | 1.649 | 0.065 | 25.4 |

The ELISA was carried out as described above, and the new buffers were used in the detection agent dilution step. Optical density (O.D.) measurements for the signal (240 ng/ml of rhuMAb2H7) and noise (10% cynomolgus monkey serum background) were calculated. The signal-to-noise ratio was also calculated for each buffer (Table 5).

High salt, metal chelating reagents such as EDTA, and detergent (CHAPS) each lowered the background, as expected. These additives also reduced the signal of rhuMAb2H7 compared to the signal obtained with parent buffer A (Table 3), but to a lesser degree that the amount of background reduction.

Overall, the additives increased the signal-to-noise ratio (Table 5). The addition of BGG into the parent buffer A, however, caused an increase in both the cynomolgus monkey serum background and the rhuMAb2H7 signal. The greater increase in the serum background using buffer A2 (the buffer containing the BGG) than in the rhuMAb2H7 signal caused the signal-to-noise ratio to drop to about 16, compared to about 20 in the parent buffer.

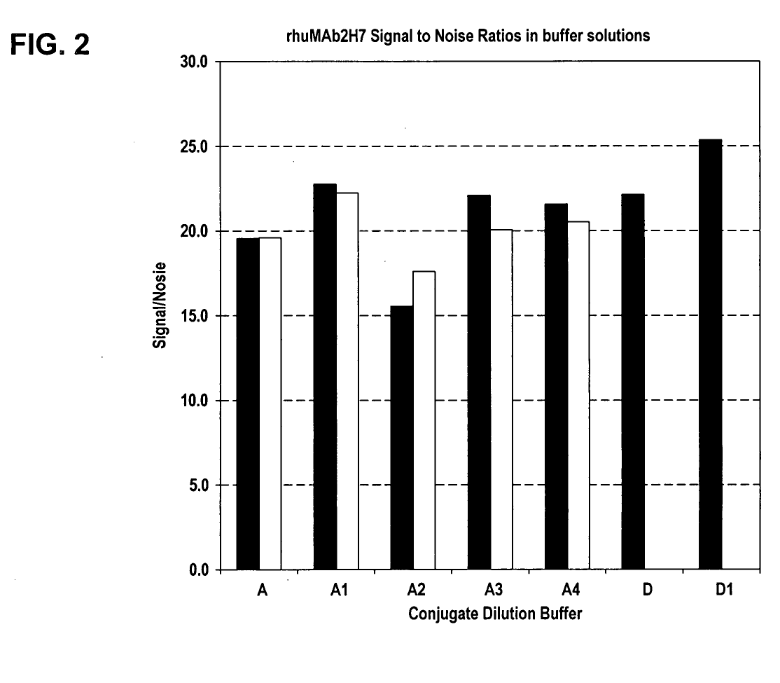

To confirm these observations, buffers A, A1, A2, A3, and A4 were diluted with an equal volume of buffer A, and then used in the detection agent dilution step. The signal-to-noise ratios were calculated for both the diluted and undiluted buffers. The results are summarized in FIG. 2, and show that buffers A1, A3, and A4 all produced an improved signal-to-noise ratio compared to parent buffer A. The undiluted buffers produced a slightly larger improvement in the signal-to-noise ratio than the diluted buffers. Diluted A2 buffer, however, produced a higher signal-to-noise ratio than undiluted buffer A2, although it was still lower than parent buffer A (FIG. 2).

These observations from the systemic evaluation of the additional additives in Buffers C and D suggested that removing BGG from the detection agent dilution step might restore the rhuMAb2H7 signal, while also improving the signal-to-noise ratio. To test this hypothesis, buffer D1 was prepared, having the same components as buffer D, except that it lacked BGG. Buffer D1 was then used in the detection agent dilution step of the ELISA assay. As expected, buffer D1 produced a much higher rhuMAb2H7 signal and signal-to-noise ratio compared to buffer D (Table 5 and FIG. 2).

Another assay was conducted to determine the effect of removing BGG from the sample diluent. The assay's sample buffer diluent contained 10% cynomolgus monkey sera and 256 ng/ml of 2H7. The ELISA was carried out as described in Example 2, but using a bridging format, where the capture reagent and the detection agent both included sheep anti-human IgG (H+L). The capture reagent was preadsorbed with monkey serum as described in Example 1. Buffer D was used for the blocking buffer and sample buffer diluent. The signal-to-noise ratio of the assay was 39 when buffer D was used as the detection agent diluent, and 44 when buffer D1 was used. Therefore, removal of BGG from the sample dilution step further enhanced the signal-to-noise ratio.

The combination of buffer D (containing BGG) in the blocking step and buffer D1 (lacking BGG) as assay diluent for each of the sample and the detection agent, was found to give the best assay performance, including a low individual cynomolgus monkey serum variation and a high signal-to-noise ratio.

### EXAMPLE 3

### Comparison of a Bridging ELISA to a Direct ELISA

The assay described in Example 2 was a Direct ELISA procedure, where the detection agent differed from the capture reagent. A Bridging ELISA format, where the detection agent and the capture reagent comprise the same agent (such as the same antibody) is known to produce a reduced non-specific background in some assays.

A Bridging format assay system was tested to compare the resulting background and background variation to that of the Direct ELISA procedure.

Comparison of cynomolgus monkey serum backgrounds and variation with different detection agents was performed in two diluents. Goat anti-human IgG (H+L)●HRP and monkey serum adsorbed sheep anti-human IgG (H+L)●HRP were each used in the direct and bridging formats, respectively. Sheep anti-human IgG (H+L) was used as a capture reagent. Buffer D was used to block the plate and to dilute the samples. The ELISA assay was carried out as described for Example 2. A rhuMAb2H7 sample was incorporated into each experiment to ensure that the positives have similar signals.

Results are shown in Table 6. Screening of the 10% serum background from six individual cynomolgus monkeys suggested that the bridging format resulted in a lower serum background, and decreased variation in individual serum backgrounds (Table 6).

The Bridging Format ELISA was repeated to determine if parent buffer A could replace the detection agent diluent D1 described in Example 2. Side-to-side comparisons suggested that even with the Bridging Format, a buffer that contained the additional additives of buffer D1 (e.g., BGG) was needed to maintain low background variation, despite the fact that buffer A gave a much lower mean cynomolgus monkey serum background (Table 6).

TABLE 6

| | | Number of | | |
|---|---|---|---|---|
| ELISA | Detection | Cynomolgus | mean | CV % of |
| Format | Diluent | Monkey Serum | background | background |
| Direct | D1 | 6 | 0.132 | 17.4 |
| Bridging | D1 | 6 | 0.040 | 3.5 |
| Bridging | D1 | 16 | 0.041 | 3.2 |
| Bridging | A | 12 | 0.009 | 33.8 |

The assay methods described above were also conducted using a direct ELISA format, where the capture reagent was sheep anti-human IgG, monkey serum adsorbed, and the detection agent was goat anti-human IgG (H+L).

Results are shown in Table 7. In this assay, use of Buffer D (containing BGG) in each of the buffers produced a CV % of 21%, compared with a range of 50%-63% when BGG was absent from one or more of the three buffers. This data suggests that when the direct ELISA format is used, BGG can be a useful component in each of the blocking buffer, sample diluent buffer, and detection agent diluent buffer. A possible explanation for the positive results observed from the use of BGG in all three buffers in a direct ELISA format is that, due to the weak interaction of BGG with the capture reagent, the BGG may be washed away if it is not included in the sample diluent buffer.

TABLE 7

| | Regular buffer | | | |
|---|---|---|---|---|
| Blocking buffer | (buffer A) | D | D1 | D |
| Sample diluent | D | D1 | D1 | D |
| Conjugate diluent | D1 | D1 | D1 | D |
| N of cyno individuals | 20 | 20 | 20 | 20 |
| CV % of 10% cyno | 51% | 63% | 50% | 21% |
| serum background | | | | |

To determine the optimal concentration of the capture reagent in the assay methods, the capture reagent was screened at concentrations from 0.25 μg/ml to 2 μg/ml in the assay. Capture reagent concentrations of 0.25 and 0.5 μg/ml produced unacceptably low response, while a concentration of 1-2 μg/ml produced good response curves. The optimal concentration of antibody, taking into consideration both high response and economic considerations, was determined to be 1 μg/ml.

### EXAMPLE 4

### Determination of Sensitivity, Accuracy, and Linearity of rhuMAb2H7 Cynomolgus Monkey Serum PK Assay

After evaluating conditions, the quality of the optimized assay, using a bridging ELISA format, buffer D as the blocking buffer, and buffer D1 as the sample and detection buffers, was analyzed using several criteria, including assay sensitivity, accuracy, and linearity.

TABLE 8

| Concentration | | | Variance Components (% CV) | | |
|---|---|---|---|---|---|
| Target | Mean | Recovery | Inter-assay | Intra-assay | Overall |
| (ng/ml) | (ng/ml) | (%) | Precision | Precision | Precision |
| 1.56 | 1.33 | 85.3% | 3.8% | 4.2% | 5.6% |
| 2.00 | 1.90 | 95.0% | 4.5% | 1.7% | 4.8% |
| 3.12 | 2.82 | 90.4% | 5.5% | 2.5% | 6.1% |
| 4.00 | 3.91 | 97.8% | 5.1% | 4.0% | 6.5% |
| 91 | 91.2 | 100.2% | 3.8% | 3.4% | 5.1% |
| 94 | 91.2 | 97.0% | 3.9% | 3.0% | 4.9% |
| 97 | 97.1 | 100.1% | 4.3% | 3.6% | 5.6% |
| 100 | 104.8 | 104.8% | 4.0% | 5.0% | 6.4% |

1. Standard Curve Range and Sensitivity:

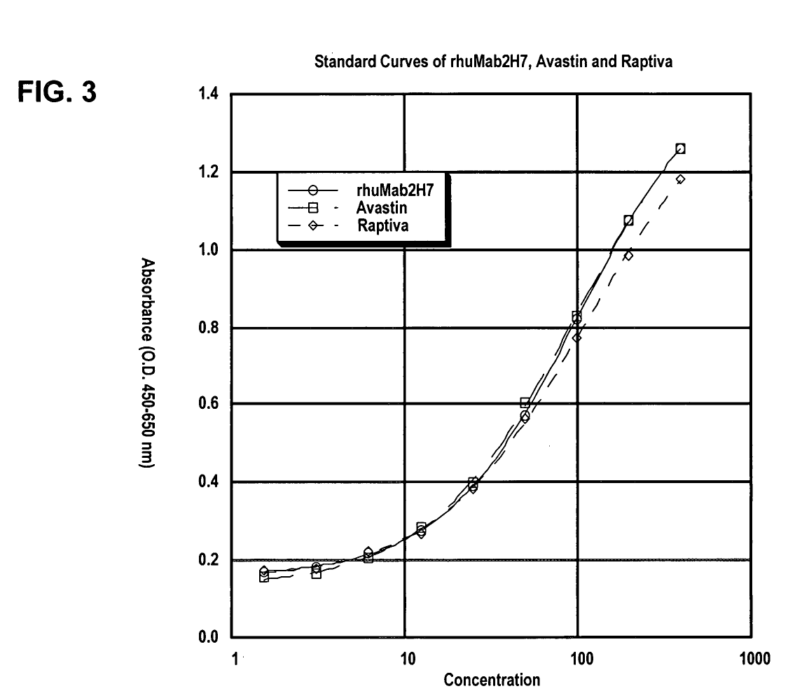

A standard curve of rhuMAb2H7 in buffer was generated in the range of 1.56 ng/nl to 400 ng/ml (FIG. 3). In order to determine both the lower and upper limits of quantification (LLOQ and ULOQ, respectively), rhuMAb2H7-spiked samples in 10% cynomolgus monkey serum were prepared at various concentrations. Aliquots were made and kept frozen to mimic the storage conditions of real samples until analysis.

Twenty samples of each concentration were analyzed over four days. The variance components of each concentration are summarized in Table 8. It was determined that the LLOQ and ULOQ were 1.56 ng/ml and 100 ng/ml, respectively, based on variance component analysis.

2. Accuracy of the Assay:

To determine the accuracy of the assay, rhuMAb2H7 at low (15.6 ng/ml), medium (300 ng/ml), and high (1000 ng/ml) concentrations was spiked into buffer or individual cynomolgus monkey serum. Samples were diluted to 1:10 and analyzed. Recovery yields of cynomolgus monkey serum samples were corrected by buffer recoveries and are summarized in Table 9. At three different concentrations tested, spike recovery of rhuMAb2H7 had a mean value of 91%, 87%, and 95%, with a CV % range from 2% to 8%.

TABLE 9

| | Target Concentrations (ng/ml) | | |
|---|---|---|---|
| Recovery | 15.6 | 300 | 1000 |
| Individual 1 | 102% | 89% | 104% |
| Individual 2 | 84% | 87% | 94% |
| Individual 3 | 89% | 87% | 87% |
| Individual 4 | 89% | 84% | 93% |
| Individual 5 | 90% | 88% | N.D. |
| Mean recovery | 91% | 87% | 95% |
| CV % | 7% | 2% | 8% |

3. Linearity of Dilution:

Real samples from PK studies would have a wide range of concentrations of rhuMAb2H7 and therefore need to be diluted in several steps to be analyzed within the assay range. To evaluate if concentrations can be accurately determined after a large dilution factor, experiments were performed to test the linearity of the assay. In this experiment, rhuMAb2H7 was spiked into individual cynomolgus monkey serum at two different concentrations (1000 and 300 ng/ml), and the dilution-corrected concentrations as determined by the experiment were compared with each sample serial dilution. The percentage difference of dilution-corrected concentration values for each serial diluted sample was less than 18%, suggesting samples were diluted linearly within the tested range.

4. Intra- and Inter-Assay Precision:

To determine both intra- and inter-assay precisions, control samples were prepared by diluting rhuMAb2H7 into neat cynomolgus monkey serum at concentrations of 30, 300, and 800 ng/ml. Replicates of each set of controls were analyzed with a freshly prepared standard curve on different plates in a same day, and the procedure was repeated for several days. Variance components (% CV) were calculated for the control samples at each concentration, and are shown in Table 10. “Intra-assay precision” refers to the CV % obtained for each concentration within a same-day experiment, while the inter-assay precision was obtained using data over several days.

TABLE 10
Summary of the intra-and inter-assay precisions

| | Concentration | | |
|---|---|---|---|
| | (ng/ml) | | |
| | 30 | 300 | 800 |
| Intra-assay precision | 3% | 5% | 5% |
| Inter-assay precision | 5% | 5% | 4% |

### EXAMPLE 5

### Applicability of the Assay to Other Antibodies

a. Use of the Assay to Generate Standard Curves for Various Humanized Antibodies.

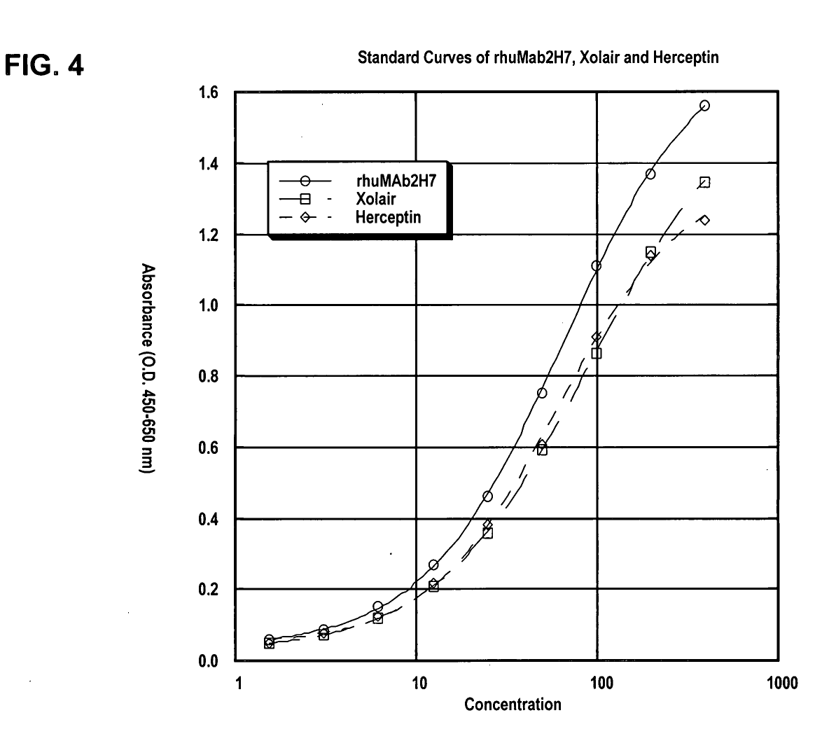

None of the reagents used in the assay described in Examples 1-4 were specific to rhuMAb2H7. To determine if the assay could be useful to quantify other humanized antibodies, the rhuMAb2H7 cynomolgus bridging ELISA PK assay described in Examples 1-4, with buffer D used as the blocking buffer D1 as the sample and detection agent buffers, was used to generate standard curves for several other humanized antibodies including AVASTIN™, RAPTIVA®, XOLAIR®, and HERCEPTIN®. Results, shown in FIGS. 3 and 4, demonstrated that all the tested antibodies were quantitated with high specificity using the assay method showed a good cross-reactivity with the assay of the invention.

b. Use of the Nontarget-Specific, Bridging ELISA Assay to Quantify Herceptin® In 1% and 10% Cynomolgus Monkey Serum.

TABLE 11

| | Target Herceptin | | Herceptin |
|---|---|---|---|
| Serum | concentration | Herceptin recovery | recovery |
| concentration | (ng/ml) | (specific assay)¹ | (non specific)² |
| 10% | 75 | 91 | 88 |
| | | 89 | 85 |
| | 25 | 90 | 84 |
| | | 93 | 90 |
| | 5 | 102 | 95 |
| | | 106 | 94 |
| 1% | 75 | 99 | 105 |
| | | 106 | 107 |
| | 25 | 106 | 105 |
| | | 104 | 100 |
| | 5 | 114 | 108 |
| | | 104 | 103 |

¹Spike recovery using Herceptin-specific assay (%).
²Spike recovery of Herceptin using rhuMAb2H7 assay (%).

In order to further test the usefulness of the assay methods described herein, the bridging ELISA assay described in Examples 1-4, with buffer D used as the blocking buffer and Buffer D1 as the sample and detection agent buffer, was tested sided by side with a target-specific assay for Herceptin, to quantify Herceptin in 1% and 10% cynomolgus monkey serum. The target-specific assay used the extracellular domain (ECD) of HER2 as the capture reagent, and goat anti-human Fc•HRP as the detection agent. Different concentrations of Herceptin were spiked into 1% and 10% cynomolgus monkey serum, and the recovery was measured with two different assays.

The results of the comparison are shown in Table 11. Both assays gave very comparable spike recoveries of Herceptin at all concentrations tested.

c. Use of Goat Anti-Human IgG to Detect mAb 2H7 In the Optimized, Bridging ELISA Format Assay of the Invention.

TABLE 12

| Assay | O.D. (2H7 conc.) | O.D.¹ | Recovery⁴ | Recovery⁴ |
|---|---|---|---|---|
| 1 | 4.08 (50 ng/ml) | 4.12² | Not determined | Not determined |
| 2 | 0.049 | 0.023³ | 80% | 104% |
| | (1.56 ng/ml of 2H7) | | | |

¹of 10% cyno serum.
²(higher than the O.D. of 50 ng/ml of 2H7).
³(lower than the O.D. of 1.56 ng/ml of 2H7).
⁴of 30 ng/ml of 2H7 in 10% cyno serum.

To test if another polyclonal anti-human IgG can replace sheep anti-human IgG as a capture reagent, a further assay was conducted using goat anti-human IgG as the capture reagent. Antisera obtained from a goat immunized with mAb 2h7 were purified against a 2H7 column and subsequently with or without a cynomolgus monkey serum protein column. The cynomolgus monkey serum background was compared in two assays, where assay #1 used reagents purified from the 2H7 column only, and assay #2 used reagents purified from both columns. Both methods used a bridging format, and the buffer systems of the optimized assay discussed in Examples 1-4 (with Buffer D used as the blocking buffer, and Buffer D1 as the sample and detection buffer). Results are summarized in Table 12. The results show that other polyclonal anti-human IgGs can successfully be used as the capture reagent in the assay of the invention.

Serum from additional species was also evaluated for the spike recovery of 100 ng/ml rhuMAb2H7 in 10% serum from rodent and other non-human primate sera. The results are summarized in Table 13.

TABLE 13

| Species | Recovery |
|---|---|
| Rat | 97% |
| Mouse | 99% |
| Baboon | 81% |
| African green | 85% |
| Rhesus monkey | 93% |

The data shows that the non-target specific assay accurately detects rhuMAb2H7 not only in cynomolgus monkey serum, but also in other sera including rat, mouse, baboon, African green monkey, and Rhesus monkey.

### EXAMPLE 6

### Comparison of the Non-Target Specific, Direct ELISA Format huMAb2H7 Cynomolgus Monkey Serum PK Assay Using BGG-Containing Buffer D as the Blocking Buffer, with Other Blocker Solutions.

TABLE 14

| | | | OD 450-650 nm |
|---|---|---|---|
| | | | for 10% |
| | Blocking buffer | Assay diluent | cyno serum |
| 1 | PBS containing 0.5% | PBS containing 0.5% BSA, 0.05% Proclin- | 0.147 |
| | BSA, 0.05% Proclin-300, | 300, and 0.05% P20, pH 7.3 | |
| | and 0.05% P20, pH 7.3 | | |
| 2 | PBS containing 2% BSA, | PBS containing 0.5% BSA, 0.05% P20, | 0.126 |
| | 0.05% Proclin-300, and | 0.05% Proclin-300, 5 mM EDTA, 0.2% | |
| | 0.05% P20, pH 7.3 | beta-gamma globulin, 0.25% CHAPS, and | |
| | | 0.35 M NaCl, pH 7.98 | |
| 3 | Same as #2 | Same as the diluent in #2 except that the pH | 0.127 |
| | | is 7.98 | |
| 4 | Same as #2 | Same as the diluent in #2 except that the pH | 0.127 |
| | | is 7.08 | |
| 5 | Same as #2 | Same as the diluent in #2 except that the pH | 0.131 |
| | | is 6.55 | |
| 6 | Same as #2 | PBS containing 0.5% BSA, 0.05% Proclin- | 0.148 |
| | | 300, and 0.05% P20, 0.1% Triton X-100, | |
| | | pH 7.3 | |
| 7 | Same as #2 | PBS containing 0.5% BSA, 0.05% Proclin- | 0.143 |
| | | 300, and 0.05% P20, 0.1% Tween-80, pH | |
| | | 7.3 | |
| 8 | Same as #2 | PBS containing 0.5% BSA, 0.05% Proclin- | 0.156 |
| | | 300, and 0.05% P20, 0.1% n-octyl-b-D- | |
| | | glucopyranoside, pH 7.3 | |
| 9 | PBS containing 0.5% | PBS containing 0.5% BSA, 0.05% Proclin- | 0.138 |
| | BSA, 0.05% Proclin-300, | 300, and 0.05% P20, pH 7.3 | |
| | and 0.05% P20, 1% | | |
| | gelatin, pH 7.3 | | |
| 10 | Same as #9 | PBS containing 0.5% BSA, 0.05% P20, | 0.092 |
| | | 0.05% Proclin-300, 5 mM EDTA, 0.2% | |
| | | beta-gamma globulin, 0.25% CHAPS, and | |
| | | 0.35 M NaCl, pH 8.90 | |
| 11 | Same as #9 | Same as the diluent in #10 except that the | 0.100 |
| | | pH is 7.98 | |
| 12 | Same as #9 | Same as the diluent in #10 except that the | 0.108 |
| | | pH is 7.08 | |
| 13 | Same as #9 | Same as the diluent in #10 except that the | 0.111 |
| | | pH is 6.55 | |
| 14 | Same as #9 | PBS containing 0.5% BSA, 0.05% Proclin- | 0.120 |
| | | 300, and 0.05% P20, 0.1% Triton X-100, | |
| | | pH 7.3 | |
| 15 | Same as #9 | PBS containing 0.5% BSA, 0.05% Proclin- | 0.120 |
| | | 300, and 0.05% P20, 0.1% Tween-80, pH | |
| | | 7.3 | |
| 16 | Same as #9 | PBS containing 0.5% BSA, 0.05% Proclin- | 0.121 |
| | | 300, and 0.05% P20, 0.1% n-octyl-b-D- | |
| | | glucopyranoside, pH 7.3 | |
| 17 | Superblock from Pierce | PBS containing 0.5% BSA, 0.05% Proclin- | 0.111 |
| | | 300, and 0.05% P20, pH 7.3 | |
| 18 | Casein from Pierce | PBS containing 0.5% BSA, 0.05% Proclin- | 0.186 |
| | | 300, and 0.05% P20, pH 7.3 | |

High concentrations of BSA and the addition of gelatin have previously been shown effective in controlling the background (Pruslin et al., 1991; Harlow et al., 1988). Therefore, in an attempt to minimize the background and the background variation with individual cynomolgus monkey serum, different conditions for both the blocking buffer and the sample/detection buffers were tested in addition to the use of BGG (as described in Example 2). These tests were conducted using a direct ELISA format, with sheep anti-human IgG (H+L) (monkey serum adsorbed) serving as the capture reagent, and goat anti-human IgG (H+L) HRP as the detection agent. The assay diluents were used to dilute both the sample and detection agent. The washing steps, incubation time, and detection steps were as described in Example 2. The results are shown in Table 14.

The data suggests that while some of the components in the tested blocking solutions may reduce the background, none of the solutions resulted in a significant reduction of the cynomolgus serum background. Therefore, it appeared that readily available buffers were not sufficient to solve the high background problem, even using the improved capture reagent (sheep anti-human IgG (H+L), monkey serum adsorbed) that had been developed. Furthermore, the commercially available block solutions Superblock and Casein (both from Pierce) also produced little improvement in the background levels. Finally, blocking solutions containing 0.1% of the detergents Triton X-100, Tween-80 and n-octyl-β-D-glucopyranoside resulted in a cynomolgus monkey serum similar to that obtained with 0.05% of Tween-20.

### EXAMPLE 7

### Use of Fish Gelatin and Mammalian IgGs In the Blocking Buffer

Further experiments were conducted to determine if BGG could be replaced in the assay methods described herein by fish gelatin or other mammalian immunoglobulins, including mouse IgG, rabbit IgG, donkey IgG. The assay was carried out similarly to the procedure described in Example 2, but using a bridging ELISA format. Sheep anti-human IgG (1 μg/ml, monkey serum preadsorbed) was used as the capture reagent. Fish gelatin or another mammalian immunoglobulin was used in the blocking buffer and/or sample buffers and detection agent buffers, in place of BGG. The results are shown in Table 15.

TABLE 15

| | Blocking buffer | | | | | | |
|---|---|---|---|---|---|---|---|
| | D | D | D | D1 + 0.2% | D1 + 0.2% | D1 + 0.2% | D1 + 0.2% |
| | | | | mouse IgG | rabbit IgG | donkey IgG | fish gelatin |
| | Sample diluent | | | | | | |
| | D | D1 | D | D1 + 0.2% | D1 + 0.2% | D1 + 0.2% | |
| | | | | mouse IgG | rabbit IgG | donkey IgG | |
| | Detection reagent diluent | | | | | | |
| | D | D1 | A | D1 + 0.2% | D1 + 0.2% | D1 + 0.2% | |
| | | | | mouse IgG | rabbit IgG | donkey IgG | |
| N | 20 | 20 | 12 | 20 | 20 | 20 | 20 |
| CV %¹ | 7% | 8% | 34% | 13% | 30% | 23% | 35% |
| Signal² | 1.15 | 1.04 | 1.6³ | 0.09 | 0.06 | 0.38 | 0.34 |
| Mean of | .015 | .015 | | | | | |
| 10% cyno | | | | | | | |
| serum. | | | | | | | |
| S/N | 76.8 | 94.2 | | | | | |

¹of 10% cyno serum background.
²Signal of 2H7 at 240 ng/ml.
³of 256 ng/ml of 2H7.

The results indicate that use of fish gelatin, donkey IgG, rabbit IgG, or mouse IgG in the blocking buffer produced substantially higher signals than that obtained in a target specific capture assay. The signal produced when these agents were used was substantially less than obtained in the use of BGG in the blocking buffer. The data also reveals that, in assays using a bridging format ELISA, the background variation as measured by the CV % increased substantially when BGG was absent from the blocking buffer (Table 15).

Also of interest is that in the bridging ELISA format, removing the BGG from the sample buffer and detection agent buffer, but maintaining it in the blocking buffer, resulted in a higher signal-to-noise ratio than when BGG was used in the blocking buffer and both buffers, even though the serum background variation (CV %) remained similar (Table 15).

### EXAMPLE 8

### Binding Affinities of CD20 Peptides and Full-Length CD20 to rhuMAb2H7

In general, a pharmacokinetic (PK) assay that quantifies the concentration of a target molecule in a body fluid such as serum requires one or more target-specific molecules to serve as the capture reagent. During the course of the present investigations, therefore, attempts were made to develop a cynomolgus rhuMAb2H7 PK assay via the use of a soluble CD20 peptide, is target-specific for rhuMAb2H7, as the capture reagent. Both the full-length CD20 polypeptide, as well as peptides that resemble the C-terminal extracellular domain of CD20, were synthesized and evaluated for their usefulness in an assay for rhuMAb2H7.

Four CD20 peptides were synthesized:

| | |
|---|---|
| (1) | |
| a disulfide-cyclized mono-biotin | [SEQ. ID. NO. 1] |
| 35 mer having the sequence Biotin- | |
| FIRAHTPYINIYNCEPANPSEKNSPSTQYCYSGG | |
| K-amide, | |
| (2) | |
| a Bis-biotin disulfide-cyclized | [SEQ. ID. NO 2] |
| 35-mer having the sequence Biotin- | |
| FIRAHTPYINIYNCEPANPSEKNSPSTQYCYSGG | |
| K (Biotin)-amide, | |
| (3) | |
| a disulfide-cyclized mono-biotin | [SEQ. ID. NO. 3] |
| 51-mer having the sequence Biotin- | |
| GKISHFLKMESLNFIRAHTPYINIYNCEPANPSE | |
| KNSPSTQYCYSIQSGGK-amide, | |
| and | |
| (4) | |
| a disulfide-cyclized Bis-biotin 51- | [SEQ. ID. NO. 4] |
| mer having the sequence Biotin GKI | |
| SHFLKMESLNFIRAHTPYINIYNCEPANPSEKNS | |
| PSTQYCYSIQSGGK (Biotin)-amide. | |

1. Synthesis of CD20 ECD Peptides.

The CD20 peptides were synthesized on rink amide resin using solid phase methods utilizing Fmoc amine protection. The synthesis was carried out in N,N-dimethylformamide on a Pioneer peptide synthesizer from ABI using 4 equivalents of amino acid, 4 equivalents HBTU and 5 equivalent Diisopropylethyl with a one hour coupling time. After two coupling cycles the Fmoc was removed using 10% piperidine in N,N-dimethylformamide. The biotin was coupled to the free nitrogens overnight using 4 equivalents of biotin dissolved in 1:1 dimethyl sulfoxide:dimethylformamide with 4 equivalents PyBOP, and 5 equivalents diisopropylethyl amine. The C-terminal biotin was attached via removing the ivDde on the ε-nitrogen on the C-terminal lysine by treatment with 5% hydrazine in DMF immediately proceeding biotin attachment. The peptides were cleaved from the resin by shaking in TFA with 5% triisopropyl silane for 1 hour. The resin was separated via filtration, and the TFA removed under reduced pressure. The peptides were precipitated with ethyl ether, and purified by HPLC chromatography using a 0-60 water: acetonitrile gradient that contained 0.1% TFA. Purified peptides were obtained as white powders after lyophilization. LCMS of the peptides produced a single peak, and revealed that the peptides had the predicted mass.

2. Expression and Purification of CD20 Full Length Molecule.

Materials

All detergents were obtained from Anatrace Inc. (Maumee, Ohio). Unless otherwise mentioned all chemicals were obtained from Sigma-Aldrich (St. Louis, Mo.). Rituximab® (C2B8) was generated as disclosed in U.S. Pat. No 5,736,137.

Cloning & Expression

The cDNA for human and murine CD20 was sub-cloned, using standard molecular biology techniques (Ausubel, et al. (Eds.), Current Protocols in Molecular Biology, John Wiley & Sons (2003)), into a BR322-derived plasmid containing the β-lactamase gene and tRNA genes for three rare E. coli codons (argU, glyT and pro2). A short MKHQHQQ sequence was added to the N-terminus of CD20 to ensure high translation initiation and an octa-His sequence was placed at the C-terminus to aid in detection and purification. Gene transcription was under control of the phoA promoter. Gene expression was induced by dilution of a saturated LB carbenicillin culture into C.R.A.P. phosphate limiting media (Simmons, et al., 2002, J. Immunol. Methods 263, 133-147) and the culture grown at 30° C. for 24 hours. Cysteine residues 111 and 220 were mutated to serines by site directed mutagenesis to improve protein behavior (C2S mutant.) Fermentor expression of CD20 was then performed (Simmons, et al., Supra).

Protein Isolation

To determine detergent extraction conditions for the his-tagged human CD20 expressed in E. coli, 5 g of cells were resuspended using a Polytron (Brinkmann, Westbury, N.Y.) in 50 mL buffer A (20 mM Tris, pH 8.0, 5 mM EDTA) and centrifuged at 125,000×g for 1 hour. The cell pellet was then resuspended in buffer A, lysed by cell disruption using a microfluidizer (Microfluidics Corp, Newton, Mass.), and centrifuged at 125,000×g for 1 hour. The pellet was washed once in the same buffer without EDTA and pelleted as before. The pellet was resuspended in 20 mL buffer B (20 mM Tris, pH 8.0, 300 mM NaCl), aliquoted and detergents were added to individual aliquots at the following concentrations: 1% SDS, 1% n-dodecyl-N,N-dimethylamine-N-oxide (LADO), 1% dodecylphosphocholine (DDPC, Fos-Choline® 12), 1% n-dodecyl-β-D-maltoside (DDM), 1% Triton-X 100 and 2.5% CHAPS. Pellets were extracted overnight at 4° C., except for the SDS sample that was extracted at room temperature. The following day the samples were centrifuged and the supernatants removed. Pellets and supernatants were re-suspended in reducing SDS loading buffer to equal volumes and analyzed by SDS-PAGE and immunoblots on nitrocellulose membranes probed with horseradish peroxidase-conjugated anti-his antibodies (Roche Applied Science, Indianapolis, Ind.).

For large-scale extraction, 100 to 200 g of cells were lysed and the insoluble fraction prepared as previously described. To extract CD20 from the insoluble fraction, the final pellet was re-suspended in buffer B at approximately 1:2.5 wt/vol from the starting wet cell weight, DDPC was added to 1% and the solution was stirred overnight at 4° C. The next day the detergent insoluble fraction was pelleted by ultracentrifugation at 125,00×g for 1 hour. The supernatant was loaded onto a Ni—NTA Superflow column pre-equilibrated with buffer B and 5 mM DDPC. The column was washed with 10 CV of buffer A with 20 mM imidazole and eluted with buffer A with 250 mM imidazole. All purification steps through column loading were performed at 4° C.

The eluant fractions containing CD20 were concentrated and loaded onto a Superdex 200 column (Amersham Biosciences, Piscataway, N.J.) pre-equilibrated in buffer A with 5 mM DDPC. The his-tagged human CD20 and murine CD20 were further purified over a 5 mL HiTrap HP Q (Amersham Biosciences, Piscataway, N.J.) column prior to gel filtration. For detergent exchange, samples were passed over a Superdex 200 column in buffer B, (0.1% DDM, 150 mM NaCl, 20 mM HEPES, pH 7.2.) Alternatively, samples were bound to a small Ni—NTA column, washed with buffer B and eluted with buffer B containing 300 mM imidazole. These samples were then dialyzed against buffer B to remove imidazole.

For affinity purification of human CD20, Rituximab® was immobilized at 6 mg/ml on 10 mL of Actigel ALD Superflow resin (Sterogene, Carlsbad, Calif.) This resin was placed in a column and equilibrated in buffer B. Human CD20 C2S mutant, purified as previously described for native hCD20, was passed over the column and unbound protein was removed by extensive washing in buffer B. Protein was eluted in 0.1% DDM, 150 mM NaCl and 20 mM sodium citrate, pH 3.5. Eluted samples were immediately neutralized, concentrated and dialyzed against buffer B. Protein concentration was determined by BCA (20) (Pierce Biotechnology, Rockford, Ill. 61101) and samples were stored at −80° C. prior to use.

Full length Rituximab® antibody was generated as disclosed in U.S. Pat. No. 5,736,137. Rituximab® Fab was expressed in E. coli and purified by Protein A and cation exchange chromatography.

The recovered full-length CD20 molecule possessed the sequence:

| | |
|---|---|
| MTTPRNSVNGTFPAEPMKGPIAMQSGPKPLFRRM | [SEQ. ID. NO. 5] |
| SSLVGPTQSFFMRESKTLGAVQIMNGLFHIALGG | |
| LLMIPAGIYAPICVTVWYPLWGGIMYIISGSLLA | |
| ATEKNSRKCLVKGKMIMNSLSLFAAISGMILSIM | |
| DILNIKISHFLKMESLNFIRAHTPYINIYNCEPA | |
| NPSEKNSPSTQYCYSIQSLFLGILSVMLIFAFFQ | |
| ELVIAGIVENEWKRTCSRPKSNIVLLSAEEKKEQ | |
| TIEIKEEVVGLTETSSQPKNEEDIEIIPIQEEEE | |
| EETETNFPEPPQDQESSPIENDSSP. | |

3. Binding Affinities of CD20 Peptides as Measured by Biacore 3000

After the CD20 peptides were synthesized, Binding Affinity to rhuMAb2H7 was measured with a biosensor system on a Biacore 3000. Two different methods were used to measure the binding affinities of CD20 peptides to rhuMAb2H7. In the first method, affinities were measured with a Biacore 3000 on a strepavidin chip using either immobilized mono-biotinylated CD20-35 mer at 95 RU, or mono-biotinylated CD20-51 mer at 94 RU, where RU is a relative surface plasmon resonance unit based upon an arbitrary scale. The running buffer was HBS-EP flowing at 50 μL/min with 25 μl of 10 mM Glycine-HCl, and a pH of 2.5 for the regeneration. 2H7-IgG was injected, and the resulting data was fit to a 1:1 binding model with Biaevaluation 3.0 software (Biacore AB, Uppsala, Sweden) to get the apparent equilibrium dissociation constants.

In the second method, affinities were measured with a Biacore 3000 on a strepavidin chip using either immobilized CD20-35 mer, bis(biotin)) at 685 RU, CD20(51-mer, mono(biotin)) at 1637 RU, or CD20(51-mer, bis(biotin)) at 1037 RU.

PBS/Tween/azide was used as the running buffer, at 20 μL/minute, with 20 mM HCl for regeneration. 2H7-IgG was injected, and the resulting data was fit to a bivalent analyte model.

TABLE 16

| | K_{D} | | Method |
|---|---|---|---|
| Mono-CD20-35mer | 41.7 | μM | 1 |
| Mono-CD20-51 mer | 130 | nM | 2 |
| Mono-CD20-51 mer | 47 | nM | 1 |
| Bis-CD20-35mer | 500 | nM | 2 |
| Bis-CD20-51mer | 110 | nM | 2 |

The results of the binding affinity studies are shown in Table 16. Because of the much smaller size of the peptides compared to the rhuMAb2H7 molecule, the only format used was where the peptide is immobilized, and the rhuMAb2H7 molecule is the analyte. Only apparent affinities were obtained with this format due to the avidity. Even with the full-length rhuMAb2H7 molecule, interactions between the peptides and the antibody were weak (Table 16). Furthermore, no detectable interactions were observed when an Ori-Tag labeled rhuMAb2H7 and biotinylated peptides were used with an electrochemiluminescence method, with concentrations up to 10 μg/ml.

TABLE 17

| | CD20 35-mer coat concentration (μg/ml) | | | | | |
|---|---|---|---|---|---|---|
| | 2 | 10 | 20 | 10 | 20 | 10 |
| Number of wash | 1 | 1 | 1 | 2 | 2 | 3 |
| cycles between | | | | | | |
| each step | | | | | | |
| O.D. (450-650 | 0.063 | 0.063 | 0.055 | 0.009 | 0.010 | 0.009 |
| nm) for 160 μg/ | | | | | | |
| ml of | | | | | | |
| rhuMAb2H7 | | | | | | |

Another ELISA assay that used a streptavidin microtiter plate to capture a biotinylated CD20 peptide was also conducted. The plate was incubated with 2-20 μg/ml of the peptide in PBS. The plate was then further blocked with regular assay diluent (PBS containing 0.5% BSA, 0.05% Proclin-300, and 0.05% P20, pH 7.3). After washing and blotting dry, 2.5-160 μg/ml of rhuMab2H7 was added to the wells, and the plate was incubated for 1 hr at room temperature. After washing and blotting dry, the plate was incubated with goat anti-human IgG (H+L) HRP conjugate for 1 hr before the washing. The results are shown in Table 17. The results show that, even with the minimum washing cycles, the signal of rhuMab2H7 is extremely low, suggesting that a streptavidin/CD20 biotinylated peptide is not suitable for use in developing a rhuMAb2H7 assay.

The weak interactions observed could be explained by the lack of a proper tertiary structure of the CD20 peptides compared to the extracellular domain (ECD) of the CD20 antigen expressed on cells. Considering the small size of the extra cellular domain of the antigen, it is likely that the cell membrane is providing some sort of anchoring support, and makes the ECD well structured. In addition, it has been shown that the disulfide bond on the ECD of the molecule is crucial for the binding activities of the antigen to its antibodies. The presence of this disulfide bond is likely involved in forming and maintaining a tertiary structure that is recognizable by several anti-CD20 antibodies, including rhuMAb2H7.

4. ELISA Using Full-Length CD20 Molecule as the Capture Reagent.

a. ELISA Format

Capture reagent (100 μl of 1 μg/ml full-length CD20) in sodium carbonate (pH 9.6) was added to a 96-well microtiter plate, and the plate was incubated at 4° C. overnight. The plate was then washed for 3 times with the washing buffer (PBS with 0.05% polysorbate-20). Blocking buffer (200 μl) was added and the plate was sealed and incubated at room temperature for 2 hours with a gentle agitation. After washing for three times followed by a blot dry, 100 μl of rhuMAb2H7 standard, controls, serum blanks and samples in the sample diluent were added to the plate that was subsequently sealed with a plate sealer. After incubating at room temperature for another hour with a gentle agitation, the plate was washed again with the washing buffer for six times and blotted dry. A volume of 100 μl of the detection agent diluted with the detection agent diluent was added and the plate was sealed and incubated at room temperature for one hour with a gentle agitation. The plate was then washed for another six times and blotted dry before the addition of 100 μl of an equal volume of TMB and H₂O₂. After incubating at room temperature for 15 minutes without agitation, the reaction was stopped by adding another 100 μl of 1 M H₃PO₄. The absorbance at 450 nm subtracted of that at 650 nm was read from the Spectra Max250 plate reader (Molecular Devices Corporation, Sunnyvale, Calif.), and the data were processed using SoftmaxPro (Molecular Devices Corporation, Sunnyvale, Calif.).

b. Standard Curve for rhuMAb2H7 Using CD20.

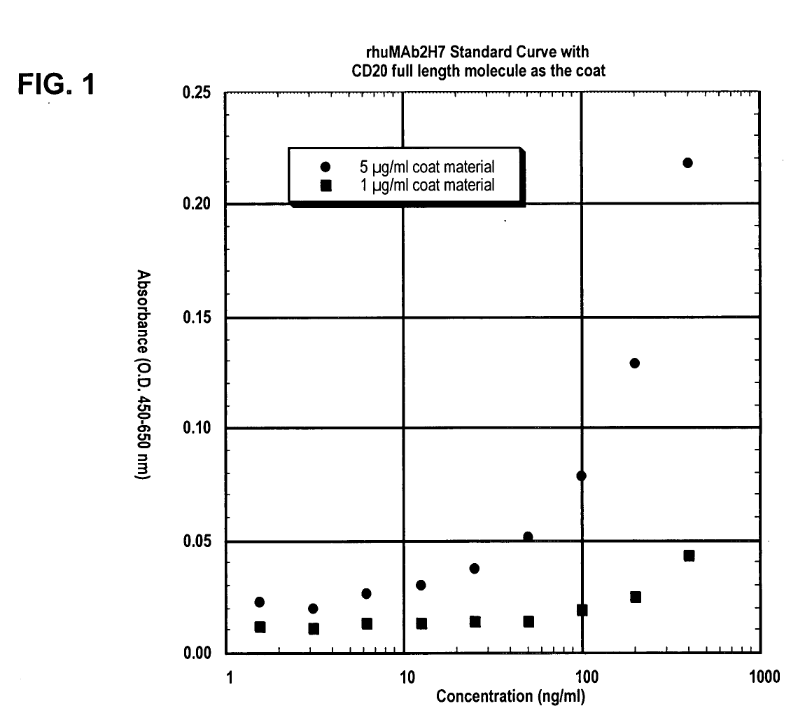

To determine the suitability of full-length CD20 as a capture reagent, rhuMAb2H7 standard curves were generated using a concentration of 1 and 5 μg/ml of the full-length CD20 molecule as the capture reagent. The signal, however, was rather weak for an antibody concentration of 400 ng/ml (FIG. 1). Screening of blank cynomolgus serum samples indicated a high background. Considering the hydrophobic characteristics of the molecule, it is not surprising that the serum background was high. In addition, the molecule was likely in an aggregated form when the molecule was coated on the microtiter plate with the CD20 extracellular domain less accessible to the rhuMAb2H7, therefore resulting in a low signal.

c. Quantification of rhuMAb 2H7 Using CD20 as the Capture Reagent.

A test using CD20 as the capture reagent in an ELISA assay for the quantification of rhuMAb 2H7 or RITUXAN® was also conducted to determine if a specific capture reagent could produce similar or even better result compared to the generic assay of the invention. The results are shown in Table 18. The low signals indicated in Table 18 show that CD20 was not useful as a capture reagent in the quantification of rhuMAb 2H7 or RITUXAN®. The applicability of the generic assay of the invention, therefore, extends even beyond the scenario where no target-specific reagents are available. The optimized generic assay of the invention may be able to quantify an antibody in cases where an assay using a target-specific capture reagent fails to produce acceptable results. This conclusion is especially significant in light of the fact that many drug targets are membrane bound antigens and, like CD20, may give similar high serum background that would reduce the effectiveness of an assay using a target-specific capture reagent.

TABLE 18

| Capture reagent | |
|---|---|
| CD20 full Length | |
| Capture reagent buffer | |
| PBS | |
| Detection agent | |
| Sheep anti-hu | |
| IgG HRP | Goat anti-hu |
| (Monkey Adsorbed) | F(ab′)2 HRP |

[Capture reagent]

| | 1 μg/mL | 5 μg/mL | 1 μg/mL | 5 μg/mL |
|---|---|---|---|---|
| N | 8 | 8 | 8 | 8 |
| mean¹ | 0.012 | 0.014 | 0.700 | 1.063 |
| signal² | 0.013 | 0.021 | 0.047 | 0.154 |
| signal³ | 0.012 | 0.023 | 0.065 | 0.166 |

¹of 10% cyno serum background (O.D.450 nm)
²of 247 ng/ml of 2H7 (O.D.450 nm)
³of 247 ng/ml of Rituxan (O.D.450 nm)

6. Discussion

CD20 has four transmembrane domains with a small extracellular domain. The intrinsic hydrophobic property of the CD20 full-length molecule contributed to a high background when cynomolgus monkey serum was used. Although the full length CD20 molecule had been used successfully in a buffer-based assay, it was not suitable for developing a cynomolgus monkey serum based PK assay with a high sensitivity. Therefore one needs to be cautious when choosing a target-specific molecule in developing an assay for a biological matrix. While the ligand of a target molecule works well in a buffer-based assay, it can potentially fail in a serum-based assay, especially when the ligand is insoluble. In the present example, relatively low affinity to rhuMAb2H7 was observed with all of the synthesized peptides.

The poor results of the CD20 binding affinities studies as discussed above highlights the need to develop an alternative assay system for quantitating antibodies sera, as disclosed in the present invention.

Although the foregoing refers to particular embodiments, it will be understood that the present invention is not so limited. It will occur to those of ordinary skill in the art that various modifications may be made to the disclosed embodiments without diverting from the overall concept of the invention. All such modifications are intended to be within the scope of the present invention.

All publications and patent applications in this specification are indicative of the level of ordinary skill in the art to which this invention pertains. All publications and patent applications are herein incorporated by reference to the same extent as if each individual publication or patent application was specifically and individually indicated by reference.

What is claimed is:

1. A method for detecting a human, human-chimeric, humanized antibody, or fragment thereof containing a constant region, in a sample containing fluid of a non-human species, comprising the steps of:
a) applying a capture reagent to an assay surface;
b) blocking non-specific binding sites of the capture reagent with a blocking buffer comprising a non-human mammalian globulin;
c) reacting the blocked capture reagent with a sample to capture antibody present in the sample; and
d) detecting captured antibodies with a detection agent comprising a detectable signal.

2. The method of claim 1, further comprising the step of:
pre-absorbing the capture reagent with non-human body fluid of the same or closely related non-human species.

3. The method of claim 1, wherein the detection agent and the capture reagent comprise the same antibody-binding moiety.

4. The method of claim 3, wherein the detection agent and capture reagent each comprises an antibody that binds a human, human-chimeric, or humanized antibody.

5. The method of claim 4, wherein the detection agent antibody comprises the capture reagent antibody.

6. The method of claim 1, wherein the detection agent comprises an antibody conjugated to a detectable label.

7. The method of claim 6, wherein the label comprises alkaline phosphatase or horseradish peroxidase.

8. The method of claim 1, wherein the sample comprises non-human primate serum.

9. The method of claim 8, wherein the capture reagent is preabsorbed with non-human primate serum of the same or closely related non-human primate species.

10. The method of claim 1, wherein the blocking buffer comprises bovine gamma globulin.

11. The method of claim 1, wherein the blocking buffer comprises mouse IgG.

12. The method of claim 1, wherein the blocking buffer comprises at least one of rabbit IgG or donkey IgG.

13. The method of claim 1, wherein the detection agent is disposed in a buffer comprising non-human mammalian globulin.

14. The method of claim 1, wherein the sample is disposed in a sample buffer and the detection agent is disposed in a detection buffer, and wherein the sample buffer, the detection buffer, or both, do not contain non-human mammalian globulin.

15. The method of claim 1, wherein the human, chimeric, humanized antibody, or fragment thereof comprises a chimeric antibody.

16. The method of claim 1, wherein the human, chimeric, humanized antibody, or fragment thereof comprises a F(ab)₂ fragment.

17. The method of claim 1, wherein the human, chimeric, humanized antibody, or fragment thereof comprises a humanized antibody.

18. The method of claim 15, wherein the antibody comprises a humanized anti-HER2 antibody.

19. The method of claim 15, wherein the antibody comprises a humanized anti-CD20 antibody.

20. The method of claim 15, wherein the antibody comprises a humanized anti-VEGF antibody.

21. A method for detecting a human, human-chimeric, humanized antibody, or fragment thereof containing a constant region, in a sample comprising non-human body fluid, comprising the steps of:
a) pre-absorbing a capture reagent comprising a non-human antibody with non-human body fluid of the same or closely related non-human species;
b) blocking non-specific binding sites of the capture reagent with a blocking buffer comprising non-human mammalian globulin;
c) reacting the blocked capture reagent with a sample to capture antibody present in the sample; and
d) detecting captured antibody with a detection agent comprising the same non-human antibody as the capture reagent;
wherein the capture reagent is disposed in a capture buffer, and the sample is disposed in a sample buffer, and wherein the capture buffer, the sample buffer, or both, does not contain the non-human mammalian globulin.

22. The method of claim 21, wherein the capture reagent is coated on an assay surface.

23. The method of claim 22, wherein said assay surface comprises a polymeric substrate, sensor chip, resin bead, or microtitre plate.

24. The method of claim 21, wherein the non-human mammalian globulin is bovine gamma globulin.

25. The method of claim 1, wherein said detecting comprises quantitating an amount of antibody in the sample.

26. The method of claim 21, wherein said detecting comprises quantitating an amount of antibody in the sample.


SEQUENCE LISTING (5 sequences)

SEQ ID NO: 1 - protein, 35 aa, Artificial
  Synthetic Soluble CD20 Peptide
FIRAHTPYINIYNCEPANPSEKNSPSTQYCYSGGK

SEQ ID NO: 2 - protein, 35 aa, Artificial; identical to SEQ ID NO: 1
  Synthetic Soluble CD20 Peptide

SEQ ID NO: 3 - protein, 51 aa, Artificial
  Synthetic Soluble CD20 Peptide
GKISHFLKMESLNFIRAHTPYINIYNCEPANPSEKNSPSTQYCYSIQSGGK

SEQ ID NO: 4 - protein, 51 aa, Artificial; identical to SEQ ID NO: 3
  Synthetic Soluble CD20 Peptide

SEQ ID NO: 5 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP